SEQUENCE LISTING (12 sequences)

SEQ ID NO: 1 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 2 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 3 - protein, 385 aa, Cannabis sativa
MNHLRAEGPASVLAIGTANPENILIQDEFPDYYFRVTKSEHMTQLKEKFRKICDKSMIRKRNIFLNEEHLKQNPKLVEHDVQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 4 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDMTACLFRGPSDSNLELLVGQAIFGDGAAAVIVGAEPDESVGERPIFELVSTGQTFLPNSEGTIGGHIREAGLMFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVLRSVPINY

SEQ ID NO: 5 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKY

SEQ ID NO: 6 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKKEKFVDSRHVLSEHGNMSSSTVLFVMDELRKRSLEEGKSTTGDGFEWGVLFGFGPGLTVETVVLRSVPINY

SEQ ID NO: 7 - protein, 399 aa, Hydrangea macrophylla
MATKSVAVEEMCKAQKAGGPATILAIGTAVPSNCYYQSEYPDFYFRVTKSDHLTDLKSKFKRMCERSSIKKRYMHLTEEILEENPNMCTFAAPSIDGRQDIVVKEIPKLAKEAASKAIKEWGQPKSNITHLVFCTTSGVDMPGCDYQLTRLLGLRPSIKRLMMYQQGCHAGGTGLRLAKDLAENNKGARVLVVCSEMTVINFRGPSEAHMDSLVGQSLFGDGASAVIVGSDPDLSTEHPLYQIMSASQIIVADSEGAIDGHLRQEGLTFHLRKDVPSLVSDNIENTLVEAFTPILMDSIDSIIDWNSIFWIAHPGGPAILNQVQAKVGLKEEKLRVSRHILSEYGNMSSACVFFIMDEMRKRSMEEGKGTTGEGLEWGVLFGFGPGFTVETIVLHSVPI

SEQ ID NO: 8 - protein, 399 aa, Hydrangea macrophylla
MATKSVAVEEMCKAQKAGGPATILAIGTAVPSNCYYQSEYPDFYFRVTKSDHLTDLKSKFKRMCDRSSIKKRYMHLTEEILKENPNMCSFAAPSIDGRQDIVVKEIPKLAKEAASKAIKEWGQPESNITHLVFCTTSGVDMPGCDYQLTRLLGLRPSIKRLMMYQQGCHAGGTGLRLAKDLAENNKGARVLVVCSEMTVINFRGPSEAHMDSLVGQSLFGDGASAVIVGSDPDLSTEHPLYQIMSASQIIVADSEGVIDGHLRQEGLTFHLRKDVPSLVSDNIENTLVEAFTPILMDSIDSIIDWNSIFWIAHPGGPAILNQVQAKVGLKEEKLRVSRHILSEYGNMSSACVFFIMDEMRKRSVEEGKGTTGEGLEWGVLFGFGPGFTVETIVLHSVPI

SEQ ID NO: 9 - protein, 399 aa, Hydrangea macrophylla
MATKSVAVEEMCKAQKAGGPATILAIGTAVPSNCYYQSEYPDFYFRVTKSDHLTDLKSKFKRMCERSSITKRYMHLTEEILEENPNMCTFAAPSIDGRQDIVVKEIPKLAKEAASKAIKEWGQPKSNITHLVFCTTSGVDMPGCDYQLTRLLGLRPSIKRLMMYQQGCHAGGTGLRLAKDLAENNKGARVLVVCSEMTVINFRGPSEAHMDSLVGQSLFGDGASAVIVGSDPDLSTEHPLYQIMSASQIIVADSEGAIDGHLRQEGLTFHLRKDVPSLVSDNIENTLVEAFTPILMDSIDSIIDWNSIFWIAHPGGPAILNQVQAKVGLKEEKLRVSRHILSEYGNMSSACVFFIMDEMRKRSVEEGKGTTGEGLEWGVLFGFGPGFTVETIVLHSVPI

SEQ ID NO: 10 - protein, 392 aa, Marchantia polymorpha
MSRSRLIAQAVGPATVLAMGKAVPANVFEQATYPDFFFNITNSNDKPALKAKFQRICDKSGIKKRHFYLDQKILESNPAMCTYMETSLNCRQEIAVAQVPKLAKEASMNAIKEWGRPKSEITHIVMATTSGVNMPGAELATAKLLGLRPNVRRVMMYQQGCFAGATVLRVAKDLAENNAGARVLAICSEVTAVTFRAPSETHIDGLVGSALFGDGAAAVIVGSDPRPGIERPIYEMHWAGEMVLPESDGAIDGHLTEAGLVFHLLKDVPGLITKNIGGFLKDTKNLVGASSWNELFWAVHPGGPAILDQVEAKLELEKGKFQASRDILSDYGNMSSASVLFVLDRVRERSLESNKSTFGEGSEWGFLIGFGPGLTVETLLLRALPLQQAERV

SEQ ID NO: 11 - protein, 101 aa, Artificial Sequence
  OAC homolog
MAVKHLIVLKFKEDITEAQKDEFFKTYVNLVNIIPAMKEVYWGKDVTAKNKDEGYTHIVEVTFESVETIQEYISHPAHVGFGDVYRSFWEKLLIFDYTPTK

SEQ ID NO: 12 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

(19) United States
(12) Patent Application Publication
(10) Pub. No.: US 2022/0177858 A1
(43) Pub. Date: Jun. 9, 2022

(54) OLIVETOL SYNTHASE VARIANTS AND METHODS FOR PRODUCTION OF OLIVETOLIC ACID AND ITS ANALOG COMPOUNDS

(71) Applicant: Genomatica, Inc., San Diego, CA (US)

(72) Inventors: Michael A. Noble, San Diego, CA (US); Russell Scott Komor, San Diego, CA (US); Jingyi Li, San Diego, CA (US)

(21) Appl. No.: 17/604,722

(22) PCT Filed: Apr. 17, 2020
(86) PCT No.: PCT/US2020/028766

§ 371 Date: Oct. 18, 2021

Related U.S. Application Data
(60) Provisional application No. 62/980,035, filed on Feb. 21, 2020.
(60) Provisional application No. 62/836,347, filed on Apr. 19, 2019.

(51) Int. Cl. C12N 9/10; C12N 15/52; C12N 1/20; C12P 7/22

(52) U.S. Cl. CPC C12N 9/1029; C12N 15/52; C12P 7/22; C12N 1/20; C12Y 203/01206

(57) ABSTRACT

Described herein are non-natural olivetol synthase (OLS) variants, nucleic acids, engineered cells, method s for preparing cannabinoids, and compositions thereof. The non-natural olivetol OLS variants form desired cannabinoid precursor and products at increased rates, have higher affinity for pathway substrates, and/or byproducts are formed in lower amounts in their presence, as compared to wild type OLS. The OLS variants can be used to form linear polyketides, and can be expressed in an engineered cell having a pathway to form cannabinoids, which include CBGA, its analogs and derivatives. CBGA can be used for the preparation of cannabigerol (CBG), which can be used in therapeutic compositions.

### CROSS REFERENCE TO RELATED APPLICATIONS

This application claims the benefit of U.S. Provisional Patent Application Ser. No. 62/836,347 filed Apr. 19, 2019, and U.S. Provisional Patent Application Ser. No. 62/980,035 filed Feb. 21, 2020, both entitled OLIVETOL SYNTHASE VARIANTS AND METHODS FOR PRODUCTION OF OLIVETOLIC ACID AND ITS ANALOG COMPOUNDS, the disclosures of which are incorporated herein by reference. The entire content of the ASCII text file entitled “GNO0107WO_Sequence_Listing.txt” created on Apr. 17, 2020, having a size of 36 kilobytes is incorporated herein by reference.

### BACKGROUND

Cannabinoids constitute a varied class of chemicals that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as cannabis, and synthetic cannabinoids, can have activity on cannabinoid receptors and elicit biological responses.

Cannabis sativa produces a variety of phytocannabinoids, for example, cannabigerolic acid (CBGA), which is a precursor of tetrahydrocannabinol (THC), the primary psychoactive compound in cannabis. Additionally, CBGA is also a precursor for Δ⁹-tetrahydrocannabinoic acid (Δ⁹-THCA), cannabichromenic acid (CBCA), and cannabidiolic acid (CBDA).

In C. sativa, precursors of cannabidiol (CBD), cannabigerol (CBG), cannabichromene (CBC), and THC are carboxylic acid-containing molecules referred to as Δ⁹-tetrahydrocannabinoic acid (Δ⁹-THCA), CBDA, cannabigerolic acid (CBGA), and cannabichromenic acid (CBCA), respectively. Δ⁹-THCA, CBDA, CBGA, and CBCA are bioactive after decarboxylation, such as caused by heating, to their bioactive forms, e.g. CBGA to CBG.

Despite the well-known actions of THC, the non-psychoactive CBD, CBG, and CBC cannabinoids also have important therapeutic uses. For example, these cannabinoids can be used for the treatment of conditions and diseases that are altered or improved by action on the CB₁ and/or CB₂ cannabinoid receptors, and/or α₂-adrenergic receptor. CBG has been proposed for the treatment of glaucoma as it has been shown to relieve intraocular pressure. CBG can also be used to treat inflammatory bowel disease. Further, CBG can also inhibit the uptake of GABA in the brain, which can decrease anxiety and muscle tension.

Cannabinoids are prenylated polyketides derived from fatty acid and isoprenoid precursors. The first enzyme in the cannabinoid pathway is a polyketide synthase, olivetol synthase (OLS), that catalyzes the condensation of hexanoyl-CoA with three molecules of malonyl-CoA to yield 3,5,7-trioxododecanoyl-CoA, which is converted to olivetolic acid (OLA) by the enzyme olivetolic acid cyclase (Gagne et al., PNAS, 109: 12811-12816). Formation of geranyl pyrophosphate stems from the mevalonate pathway (MVA) or methylerythritol-4-phosphate (MEP) pathway (also known as the deoxyxylulose-5-phosphate pathway), which produce isopentyl pyrophosphate (IPP) and dimethylallyl pyrophosphate (DMAPP), which are converted to geranyl pyrophosphate (GPP) using geranyl pyrophosphate synthase. A prenyltransferase converts OLA and GPP to CBGA, the common precursor to cannabinoids.

### SUMMARY

Aspects of the disclosure are directed towards non-natural olivetol synthases that include at least one amino acid variation that differs from an amino acid residue of a wild type olivetol synthase, engineered cells comprising the non-natural olivetol synthases, and methods of using the non-natural olivetol synthases and the engineered cells. These non-natural olivetol synthases are capable of producing precursors for prenylated aromatic compounds, including cannabinoids, analogs and derivatives thereof.

In one aspect, provided is a non-natural olivetol synthase (OLS) comprising at least one amino acid variation as compared to a wild type olivetol synthase, wherein the non-natural olivetol synthase: (a) forms olivetolic acid or olivetol from malonyl-CoA and hexanoyl-CoA at a greater rate as compared to the wild type olivetol synthase; (b) has a higher affinity for hexanoyl-CoA and/or other acyl-CoA substrates as compared to the wild type olivetol synthase; (c) forms olivetolic acid analogs, olivetol analogs, variants thereof, or combinations thereof from malonyl-CoA and other acyl-CoAs at a greater rate as compared to the wild type olivetol synthase; (d) is characterized by a lower amount of one or more pyrone-based compounds being formed in the presence of the non-natural olivetol synthase (OLS) as compared to the wild type olivetol synthase, or (e) any combination of (a), (b), (c) or (d), wherein olivetolic acid or olivetol, analogs thereof, variants thereof, or acid derivatives of a polyketide are formed in the presence of olivetolic acid cyclase (OAC) which is not rate limited by amount or activity.

In one aspect, provided are nucleic acids encoding a non-natural olivetol synthase (OLS) comprising at least one amino acid variation as compared to a wild type olivetol synthase. The nucleic acid encodes an OLS that comprise at least one amino acid variation as compared to a wild type olivetol synthase, wherein the non-natural olivetol synthase: (a) forms olivetolic acid or olivetol from malonyl-CoA and hexanoyl-CoA at a greater rate as compared to the wild type olivetol synthase; (b) has a higher affinity for hexanoyl-CoA and/or other acyl-CoA substrates as compared to the wild type olivetol synthase; (c) forms olivetolic acid analogs, olivetol analogs, variants thereof, or combinations thereof from malonyl-CoA and other acyl-CoA at a greater rate as compared to the wild type olivetol synthase; (d) is characterized by a lower amount of one or more pyrone-based compounds being formed in the presence of the non-natural olivetol synthase (OLS) as compared to the wild type olivetol synthase, or (e) any combination of (a), (b), (c) or (d), wherein olivetolic acid or olivetol, analogs thereof, variants thereof, or acid derivatives of a polyketide are formed in the presence of olivetolic acid cyclase (OAC) which is not rate limited by amount or activity.

In some embodiments, the nucleic acid is operably linked to a regulatory element. In some embodiments, the regulatory element is heterologous to the olivetol synthase. In one aspect, provided are engineered cells comprising a non-natural olivetol synthase comprising at least one amino acid variation as compared to a wild type olivetol synthase. In the engineered cell, the non-natural olivetol synthase (OLS) comprises at least one amino acid variation as compared to a wild type olivetol synthase, wherein the non-natural olivetol synthase: (a) forms olivetolic acid or olivetol from malonyl-CoA and hexanoyl-CoA at a greater rate as compared to the wild type olivetol synthase; (b) has a higher affinity for hexanoyl-CoA and/or other acyl-CoA substrates as compared to the wild type olivetol synthase; (c) forms olivetolic acid analogs, olivetol analogs, variants thereof, or combinations thereof from malonyl-CoA and other acyl-CoA at a greater rate as compared to the wild type olivetol synthase; (d) is characterized by a lower amount of one or more pyrone-based compounds being formed in the presence of the non-natural olivetol synthase (OLS) as compared to the wild type olivetol synthase, or (e) any combination of (a), (b), (c) or (d), wherein olivetolic acid or olivetol, analogs thereof, variants thereof, or acid derivatives of a polyketide are formed in the presence of olivetolic acid cyclase (OAC) which is not rate limited by amount or activity.

In some embodiments, the non-natural olivetol synthase is characterized by a lower amount of one or more pyrone-based compounds being formed in the presence of the non-natural olivetol synthase (OLS) as compared to the wild type olivetol synthase The lower amount can be reflected by ratio of compounds formed in the presence of the non-natural OLS, such as the ratio of (a) a polyketide or acid derivative thereof to (b) the pyrone-based compounds(s) that is greater than the corresponding ratio formed in the presence of the wild type olivetol synthase. For example, in the presence of the non-natural olivetol synthase an amount (mol) of polyketide or acid derivative thereof to a pyrone-based hydrolysis product(s) formed can be about 1.1-fold or greater, about 1.2-fold, about 1.3-fold, about 1.4-fold, about 1.5-fold, about 1.6-fold, about 1.8-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, or about 3.0-fold or greater than the ratio (mol) formed in the presence of the wild type olivetol synthase.

In some embodiments, the non-natural olivetol synthase provides a combination of properties. For example, the non-natural olivetol synthase can form olivetolic acid or olivetol from malonyl-CoA and hexanoyl-CoA at a greater rate as compared to the wild type olivetol synthase, and/or can form olivetolic acid analogs, olivetol analogs, variants thereof, or combinations thereof from malonyl-CoA and other acyl-CoAs at a greater rate as compared to the wild type olivetol synthase; and also can form one or more pyrone-based hydrolysis product(s) at a rate that is less than the wild type olivetol synthase.

In some embodiments, the engineered cell comprises enzymes for the olivetolic acid pathway. In some embodiments, the olivetolic acid pathway comprises olivetol synthase and olivetolic acid cyclase. In some embodiments, the amino acid sequence of olivetolic acid cyclase is at least 60%, 65%, 70%, 75%, 80%, 85%, 90%, 95%, 99%, or identical to at least 25 contiguous amino acids of any one of SEQ ID NO: 11 and SEQ ID NO: 12. In some embodiments, the amino acid sequence of olivetolic acid cyclase comprises one or more amino acid substitutions as compared to any one of SEQ ID NO: 11 and SEQ ID NO: 12. In some embodiments, the amino acid sequence of olivetolic acid cyclase is SEQ ID NO: 11 or SEQ ID NO: 12. In some embodiments, the engineered cell comprises enzymes for the geranyl pyrophosphate pathway. In some embodiments, the geranyl pyrophosphate pathway comprises geranyl pyrophosphate synthase. In some embodiments, the geranyl pyrophosphate pathway comprises a mevalonate (MVA) pathway, a non-mevalonate (MEP) pathway, an alternative non-MEP or non-MVA geranyl pyrophosphate pathway using isoprenol, prenol, or geraniol as a precursor, or a combination thereof. Various pathways for generating geranyl pyrophosphate are disclosed in PCT publication WO2017161041, which is incorporated herein by reference in its entirety. Exemplary alternative non-MEP, nor MVA geranyl pyrophosphate pathways using isoprenol or prenol as a precursor are shown in FIGS. 6 and 7, respectively. Exemplary MVA and MEP pathways are shown in FIG. 8. In some embodiments, the engineered cell further comprises an exogenous nucleic acid encoding geranyl pyrophosphate synthase.

In some embodiments, the engineered cell comprises one or more exogenous nucleic acids, wherein at least one exogenous nucleic acid encodes the non-natural olivetol synthase. In some embodiments, the engineered cell comprises two or more exogenous nucleic acids, and wherein at least one exogenous nucleic acid encodes the non-natural olivetol synthase and another exogenous nucleic acid encodes olivetolic acid cyclase.

In some embodiments, the cell is a prokaryote or a eukaryote. In some embodiments, the cell is a eukaryote selected from the group consisting of yeast, fungi, plant, microalgae, and algae. In some embodiments, the cell is a prokaryote selected from the group consisting of Escherichia, Cyanobacteria, Corynebacterium, Bacillus, Ralstonia, and Staphylococcus.

In some embodiments, the engineered cell produces olivetolic acid, cannabigerolic acid (CBGA), cannabichromene (CBC), cannabichromenic acid (CBCA), cannabigerol (CBG), cannabigerolic acid(CBGA), cannabidiol (CBD), cannabidiolic acid(CBDA), cannabigerol (CBG), Δ9-trans-tetrahydrocannabinol (Δ9-THC), Δ9-tetrahydrocannabinolic acid(THCA), analogs or derivatives thereof, or a combination thereof, in which the cell produces lesser: olivetol, olivetol analogs, derivatives of olivetol, pentyl diacetic acid lactone (PDAL), hexanoyl triacetic acid lactone (HTAL), a lactone analog or derivatives thereof, or a combination thereof, as compared to a wild-type non-engineered cell or an engineered cell comprising the wild-type olivetol synthase. In some embodiments, the engineered cell does not comprise Δ9-tetrahydrocannabinolic acid (THCA) synthase and does not convert CBGA to THCA and/or THC.

In some embodiments, the engineered cell, engineered cell extract, or engineered cell culture medium comprises olivetol or analogs and derivatives of olivetol, pentyl diacetic acid lactone (PDAL), hexanoyl triacetic acid lactone (HTAL), or lactone analog or derivatives thereof, or a combination thereof, at a concentration of no more than about 50% to about 0.0001%, no more than about 20% to about 0.001%, no more than about 10% to about 0.01% by weight of the engineered cell, engineered cell extract, or engineered cell culture medium.

In some embodiments, the engineered cell, engineered cell extract, or engineered cell culture medium comprises olivetol or analogs and derivatives of olivetol, pentyl diacetic acid lactone (PDAL), hexanoyl triacetic acid lactone (HTAL), or lactone analog or derivatives thereof, or a combination thereof, at a concentration of about 50%, 45%, 40%, 35%, 30%, 25%, 20%, 15%, 12.5%, 10%, 7.5%, 5%, 2.5%, 1%, 0.1%, 0.05%, 0.01%, 0.005%, 0.001%, 0.0005%, or 0.0001% by weight of the engineered cell, engineered cell extract or engineered cell culture medium. In some embodiments, the engineered cell further optionally includes one or more additional metabolic pathway gene(s) for generation of cannabinoid, cannabinoid analogs or derivatives, precursors of cannabinoid, cannabinoid precursors, analogs, or derivatives, or to improve recovery of the cannabinoid or its analogs or derivatives from the engineered cell.

In some embodiments, the engineered cell, engineered cell extract, or engineered cell culture medium comprises olivetolic acid, analogs or derivatives thereof, or a combination thereof, at a concentration of 50% or greater of the total products of non-natural olivetol synthase catalyzed reactions in combination with the activity of olivetolic acid cyclase (OAC).

In one aspect, provided are method for forming an aromatic compound, comprising: (a) contacting three molecules of malonyl-CoA and an acyl-CoA substrate with a non-natural olivetol synthase of the disclosure, wherein the non-natural olivetol synthase preferentially produces polyketides, analogs and derivatives thereof, or combinations thereof; (b) contacting the polyketides, analogs and derivatives thereof, or combinations thereof with an olivetolic acid cyclase (OAC) enzyme, wherein the contacting forms the aromatic compound. In some embodiments, the aromatic compound is olivetolic acid, analogs and derivatives thereof, or combinations thereof.

In one aspect, provided are methods for forming a cannabinoid, an analog or derivatives thereof, or a combination thereof, comprising: (a) contacting three molecules of malonyl-CoA and an acyl-CoA substrate with a non-natural olivetol synthase in which the non-natural olivetol synthase comprises at least one amino acid variation as compared to a wild type olivetol synthase, and the non-natural olivetol synthase preferentially produces polyketides, analogs and derivatives thereof, or combinations thereof; (b) contacting the polyketides, analogs and derivatives thereof, or combinations thereof with an olivetolic acid cyclase (OAC) enzyme in which the contacting forms the olivetolic acid, analogs and derivatives thereof, or combinations thereof; (c) converting the olivetolic acid, analogs and derivatives thereof, or combinations thereof to the cannabinoid, the analog or derivatives thereof, or the combination thereof chemically or enzymatically, or by a combination of the both.

In some embodiments, the step of contacting with a non-natural olivetol synthase occurs in an engineered cell. In some embodiments, the step of converting the olivetolic acid, analogs and derivatives thereof, or combinations thereof occurs in the engineered cell.

In some embodiments, the method further comprises a step of isolating or purifying the cannabinoid, analogs and derivatives thereof, or combinations thereof from the reaction mixture. In some embodiments, the step of isolating or purifying comprises one or more of liquid-liquid extraction, pervaporation, evaporation, filtration, membrane filtration, reverse osmosis, nanofiltration, ultrafiltration, microfiltration, membrane filtration with diafiltration, membrane separation, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration.

In some embodiments, the amino acid sequence of olivetolic acid cyclase is at least 60%, 65%, 70%, 75%, 80%, 85%, 90%, 93%, 94%, 95%, 96%, 97%, 98%, or 99% identical to at least 25, 30, 35, 40, 45, 50, 55, 60, 65, 70, 75, 80, 85, 90, or 95 or more contiguous amino acids of any one of SEQ ID NO: 11 and SEQ ID NO: 12. In some embodiments, the amino acid sequence of olivetolic acid cyclase comprises one or more amino acid substitutions as compared to any one of SEQ ID NO: 11 and SEQ ID NO: 12. In some embodiments, the amino acid sequence of olivetolic acid cyclase is SEQ ID NO: 11 or SEQ ID NO: 12. In some embodiments, one or more amino acids selected from His5, Ile7, Leu9, Phe23, Phe24, Tyr27, Val28, Leu30, Val40, Val59, Tyr72, Ile73, His78, Phe81, Gly82, Trp89, Leu92, and Ile94 of SEQ ID NO: 12 can be substituted with suitable amino acids. In some embodiments wherein olivetolic acid, an analog, or a derivative thereof is formed, the OAC is present in the engineered cell or in an in vitro reaction in a non-rate limiting amount or enzymatic form. In some embodiments, the OAC is present in the engineered cell or in an in vitro reaction in molar excess of OLS. In some embodiments, the molar ratio of OLS to OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more. In some embodiments the OAC is present in a form in which its activity does not limit the formation of OLA, for example the OAC is a non-natural OAC having higher activity than the wild type OAC. In embodiments the rate of formation of 3,5,7 tri-oxo acyl CoA by: OLS is same as the rate of formation of OLA by OAC. In some embodiments, the rate of formation of OLA is greater than the rate of formation of 3,5,7 tri-oxo acyl CoA

In some embodiments, depending on the starter acyl-CoA substrate, the non-naturally OLS enzyme in the presence of OAC enzyme can produce olivetolic acid or its analogs and derivatives, or without an OAC enzyme, OLS can produce olivetol or its analogs and derivatives, with three molecules of malonyl-CoA both inside an engineered cell and also in in vitro reactions using either purified enzymes or extracts of the engineered cells. For example, using hexanoyl-CoA and three molecules of malonyl-CoA, the product can be olivetolic acid or olivetol; using butyryl-CoA, the product can be divarinolic acid or divarinol (5-propylbenzene-1,3-diol; 5-propylresorcinol); starting with acetyl-CoA, the product can be orsellinic acid or orcinol (5-methylbenzene-1,3-diol; 5-methylresorcinol). Structures of the exemplary products of olivetolic acid, orsellinic acid, and divarinolic acid are shown in FIG. 9.

In some embodiments, analogs of olivetolic acid and olivetol include but are not limited to compounds described in International Patent Application publications WO2011127589A1 (e.g. 2,4-dihydroxy-6-heptylbenzoic acid), WO2018209143A1 (e.g. 2-alkyl-4,6-dihydroxy benzoic acid), divarinic acid (i.e., 2-propyl-4,6-dihydroxybenzoic), substituted resorcinols, for example, 5-methylresorcinol, 5-ethylresorcinol, 5-propylresorcinol, 5-butylresorcinol, 5-hexylresorcinol, 5-heptylresorcinol, 5-octylresorcinol, and 5-nonylresorcinol, and WO2018200888A1 (e.g. olivetolic acid analogs synthesized using CoA compounds). Each of the International Patent Application publications WO2011127589A1, WO2018209143A1, and WO2018200888A1 are incorporated herein by reference in their entireties.

The general structure of the acyl-CoA substrate for the OLS enzyme is shown in FIG. 4 as the starter molecule, where R₁ is a fatty acid side chain optionally comprising one or more functional and/or reactive groups as disclosed herein (i.e., an acyl-CoA compound analog or derivative).

In some embodiments, analogs or derivatives of: an acyl-CoA (e.g., hexanoyl-CoA), a cannabinoid, or a cannabinoid precursor (e.g., an olivetolic acid derivative) that are produced by an engineered cell disclosed herein or in a cell-free reaction mixture comprise one or more functional and/or reactive groups.

In some embodiments, the functional groups may include, but are not limited to, azido, halo (e.g., chloride, bromide, iodide, fluorine), methyl, alkyl (including branched and linear alkyl groups), alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

In some embodiments, the suitable reactive groups may include, but are not necessarily limited to, azide, carboxyl, carbonyl, amine, (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Suitable molecules of interest may include, but are not limited to, a detectable label; imaging agents; a toxin (including cytotoxins); a linker; a peptide; a drug (e.g., small molecule drugs); a member of a specific binding pair; an epitope tag; ligands for binding by a target receptor; tags to aid in purification; molecules that increase solubility; molecules that enhance bioavailability; molecules that increase in vivo half-life; molecules that target to a particular cell type; molecules that target to a particular tissue; molecules that provide for crossing the blood-brain barrier; molecules to facilitate selective attachment to a surface; and the like.

In some embodiments, the functional and reactive groups may be optionally substituted with one or more additional functional or reactive groups.

In some embodiments, the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA. In some embodiments, the other acyl-CoA substrates are one or more of C12, C14, C16, C18, C20 or C22 chain length fatty acid CoA, and an aromatic acid CoA, for example benzoic, chorismic, phenylacetic, and phenoxyacetic acid CoA.

In one aspect, provided are compositions comprising a cannabinoid, analogs or derivatives thereof, or combinations thereof obtained from an engineered cell in which the engineered cell comprises a non-natural olivetol synthase in which the non-natural olivetol synthase comprises at least one amino acid variation as compared to a wild type olivetol synthase. The composition can comprise a pyrone-based compound such as pentyl diacetic acid lactone (PDAL), hexanoyl triacetic acid lactone (HTAL), a lactone analog, or a combination thereof at a concentration of no more than about 0.1% to about 0.01% by weight.

In some embodiments, the cannabinoid is olivetolic acid, cannabigerolic acid (CBGA), cannabichromene (CBC), cannabichromenic acid (CBCA), cannabigerol (CBG), cannabigerolic acid (CBGA), cannabidiol (CBD), cannabidiolic acid (CBDA), cannabigerol (CBG), Δ9-tetrahydrocannabinolic acid(THCA), Δ9-tetrahydrocannabinol (THC), analogs or derivatives thereof, or a combination thereof. In some embodiments, the cannabinoid is cannabigerolic acid (CBGA), cannabigerol, analogs or derivatives thereof, or a combination thereof.

In some embodiments, the composition comprises CBGA, CBG, analogs or derivatives thereof at a concentration of 60% or greater of total cannabinoid compound(s) in the composition. In some embodiments, the composition further comprises at least one pharmaceutically acceptable excipient selected from the group consisting of a diluent, a binder, a lubricant, a disintegrant, a flavoring agent, a coloring agent, a stabilizer, a surfactant, a glidant, a plasticizer, a preservative, an essential oil, a humectant, an absorption accelerator, a wetting agent, an absorber, and a buffering agent.

In some embodiments, the composition is an edible, a pharmaceutical, personal care product, or a cosmetic, such as a composition for enhancing health, wellness, personal care, or beauty. In some embodiments, the composition is an edible composition in the form of a solid, solid infused with the composition, or a liquid. In some embodiments, the composition is a cosmetic in the form of a lotion, cream, or shampoo.

In some embodiments of the above aspects, the non-natural olivetol synthase preferentially produces polyketides over a pyrone-based compound such as pentyl diacetic acid lactone (PDAL), hexanoyl triacetic acid lactone (HTAL), a lactone analog, or a combination thereof, as compared to the wild type olivetol synthase.

In some embodiments of the above aspects, the non-natural olivetol synthase has higher affinity for other acyl-CoA substrates besides hexanoyl-CoA as compared to the wild type olivetol synthase. In some embodiments, the other acyl-CoA substrates are fatty acyl-CoA other than hexanoyl-CoA. In some embodiments, the other acyl-CoA substrates are one or more of acetyl-CoA, propionyl-CoA, butyryl-CoA, valeryl-CoA, heptanoyl-CoA, octanoyl-CoA, nonanoyl-CoA, or decanoyl-CoA. In some embodiments, the other acyl-CoA substrates are one or more of C12, C14, C16, C18, C20 or C22 chain length fatty acyl CoA, and an aromatic acid CoA, for example benzoic, chorismic, phenylacetic and phenoxyacetic acid CoA.

In some embodiments of the above aspects, the non-natural olivetol synthase is enzymatically capable of forming olivetolic acid, its analogs or its derivatives from malonyl-CoA and acyl-CoA in the presence of olivetolic acid cyclase (OAC), or olivetol, its analogs or its derivatives from malonyl-CoA and acyl-CoA in the absence of OAC, at a rate of least 1.01-fold greater as compared to the rate provided by the wild type olivetol synthase. In some aspects the rate is at least 1.02-fold, 1.03-fold, 1.04-fold, 1.05-fold, 1.06-fold, 1.07-fold, 1.08-fold, 1.09-fold, 1.1-fold, 1.12-fold, 1.14-fold, 1.16-fold, 1.18-fold, 1.2-fold, 1.24-fold, 1.28-fold, 1.32-fold, 1.36-fold, or 2-fold greater as compared to the rate of wild type olivetol synthase under the same reaction conditions. In some embodiments, the non-natural olivetol synthase is enzymatically capable of forming its analogs or its derivatives from malonyl-CoA and acyl-CoA in the presence of olivetolic acid cyclase (OAC) enzyme, or olivetol, its analogs or its derivatives from malonyl-CoA and acyl-CoA in the absence of OAC, at a rate of least twenty-fold greater rate as compared to the rate provided by the wild type olivetol synthase. In some embodiments, the acyl-CoA is hexanoyl-CoA and the product generated by OLS and OAC enzymes is olivetolic acid, or in the absence of OAC olivetol is generated. In some embodiments of the above aspects, the non-natural olivetol synthase has lower affinity for 3,5,7 trioxododecyl-CoA and 3,5,7 trioxododecanoate, and analogs thereof as substrates as compared to the wild type olivetol synthase.

In some embodiments of the above aspects, the non-natural olivetol synthase comprises at least two amino acid variations as compared to a wild type olivetol synthase. In some embodiments, the non-natural olivetol synthase comprises at least three, four, five, or more amino acid variations as compared to a wild type olivetol synthase.

In some embodiments of the above aspects, the wild type olivetol synthase comprises, or consists of, the amino acid sequence of any one of SEQ ID NOs: 1-10.

In some embodiments of the above aspects, the amino acid sequence of the non-natural olivetol synthase has at least about 50%, 60%, 65%, 70%, 75%, 80%, 85%, 90%, 93%, 94%, 95%, 96%, 97%, 98%, or 99%, or greater sequence identity to at least 25 contiguous amino acids of any one of SEQ ID NOs: 1-10. In some embodiments, the amino acid sequence of the non-natural olivetol synthase has at least about 90% or greater identity to at least 25 contiguous amino acids of any one of SEQ ID NOs:1-10.

In some embodiments, the amino acid sequence of the non-natural olivetol synthase comprises one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO:1. In some embodiments, the non-natural olivetol synthase comprises a single amino acid substitutions at a position selected from the group consisting of: Q82S, P131A, I186F, M187S, S195K, S195M, S197V, T293E, K314D, and K314M.

In some embodiments non-natural olivetol synthase comprises two, or more than two amino acid substitutions, selected from: (i) Q82S and P131A, (ii) Q82S and M187S, (iii) Q82S and S195K, (iv) Q82S and S195M, (v) Q82S and S197V, (vi) Q82S and K314D, (vii) P131A and I186F, (viii) P131A and M187S, (ix) P131A and S195M, (x) P131A and S197V, (xi) P131A and K314D, (xii) P131A and K314M, (xiii) I186F and M187S, (xiv) I186F and S195K, (xv) I186F and S195M, (xvi) I186F and T239E, (xvii) I186F and K314D, (xviii) M187S and S195K, (xix) M187S and S195M, (xx) M187S and S197V, (xxi) M187S and T239E, (xxii) M187S and K314D, (xxiii) M187S and K314M, (xxiv) S195K and S197V, (xxv) S195M and S197V, (xxvi) S195M and T239E, (xxvii) S195K and K314D, (xxviii) S195K and K314M, (xxix) S195M and K314D, (xxx) S195M and K314M, (xxxi) S197V and T239E, (xxxii) S197V and K314M, (xxxiii) T239E and K314D, (xxxiv) T239E and K314M, (xxxv) Q82S and I186F, (xxxvi) Q82S and T239E, (xxxvii) Q82S and K314M, (xxxviii) I186F and S197V (xxxix) I186F and K314M, (xl) S195K and T239E, (xli) S197V and K314D, (xlii) P131A and T239E, and (xliii) P131A and S195K.

In embodiments non-natural olivetol synthase comprises three, or more than three, amino acid substitutions selected from: (i) Q82S, P131A, and I186F, (ii) Q82S, P131A, and M187S, (iii) Q82S, P131A, and S195K, (iv) Q82S, P131A, and S195M, (v) Q82S, P131A, and S197V, (vi) Q82S, P131A, and T239E, (vii) Q82S, P131A, and K314D, (viii) Q82S, P131A, and K314M, (ix) Q82S, I186F, and M187S, (x) Q82S, I186F, and S195M, (xi) Q82S, I186F, and S197V, (xii) Q82S, I186F, and T239E, (xiii) Q82S, I186F, and K314D, (xiv) Q82S, I186F, and K314M, (xv) Q82S, M187S, and S195K, (xvi) Q82S, M187S, and S195M, (xvii) Q82S, M187S, and S197V, (xviii) Q82S, M187S, and T239E, (xix) Q82S, M187S, and K314D, (xx) Q82S, M187S, and K314M, (xxi) Q82S, S195K, and S197V, (xxii) Q82S, S195M, and S197V, (xxiii) Q82S, S195K, and K314D, (xxiv) Q82S, S195K, and K314M, (xxv) Q82S, S195M, and K314D, (xxvi) Q82S, S195M, and K314M, (xxvii) Q82S, S197V, and T239E, (xxviii) Q82S, S197V, and K314D, (xxix) Q82S, S197V, and K314M, (xxx) Q82S, T239E, and K314D, (xxxi) Q82S, T239E, and K314M, (xxxii) P131A, I186F, and M187S, (xxxiii) P131A, I186F, and S195K, (xxxiv) P131A, I186F, and S195M, (xxxv) P131A, I186F, and S197V, (xxxvi) P131A, I186F, and K314D, (xxxvii) P131A, I186F, and K314M, (xxxviii) P131A, M187S, and S195K, (xxxix) P131A, M187S, and S195M, (xl) P131A, M187S, and S197V, (xli) P131A, M187S, and T239E, (xlii) P131A, M187S, and K314D, (xliii) P131A, S195M, and S197V, (xliv) P131A, S195M, and T239E, (xlv) P131A, S195K, and K314D, (xlvi) P131A, S195K, and K314M, (xlvii) P131A, S195M, and K314D, (xlviii) P131A, S195M, and K314M, (xlix) P131A, S197V, and T239E, (l) P131A, S197V, and K314D, (li) P131A, S197V, and K314M, (lii) P131A, T239E, and K314D, (liii) P131A, T239E, and K314M, (liv) I186F, M187S, and S195K, (lv) I186F, M187S, and S195M, (lvi) I186F, M187S, and S197V, (lvii) I186F, M187S, and K314M, (lviii) I186F, S195K, and S197V, (lix) I186F, S195M, and S197V, (lx) I186F, S195K, and T239E, (lxi) I186F, S195M, and T239E, (lxii) I186F, S195K, and K314D, (lxiii) I186F, S195K, and K314M, (lxiv) I186F, S195M, and K314D, (lxv) I186F, S195M, and K314M, (lxvi) I186F, S197V, and T239E, (lxvii) I186F, S197V, and K314D, (lxviii) I186F, S197V, and K314M, (lxix) I186F, T239E, and K314M, (lxx) M187S, S195K, and S197V, (lxxi) M187S, S195M, and S197V, (lxxii) M187S, S195K, and T239E, (lxxiii) M187S, S195M, and T239E, (lxxiv) M187S, S195K, and K314D, (lxxv) M187S, S195K, and K314M, (lxxvi) M187S, S195M, and K314D, (lxxvii) M187S, S195M, and K314M, (lxxviii) M187S, S197V, and T239E, (lxxix) M187S, S197V, and K314D, (lxxx) M187S, S197V, and K314M, (lxxxi) M187S, T239E, and K314D, (lxxxii) M187S, T239E, and K314M, (lxxxiii) S195K, S197V, and T239E, (lxxxiv) S195M, S197V, and T239E, (lxxxv) S195K, S197V, and K314D, (lxxxvi) S195K, S197V, and K314M, (lxxxvii) S195M, S197V, and K314D, (lxxxviii) S195M, S197V, and K314M, (lxxxix) S195K, T239E, and K314D, (xc) S195K, T239E, and K314M,(xci) S195M, T239E, and K314D, (xcii) S195M, T239E, and K314M, and (xciii) S197V, T239E, and K314M.

In some embodiments of the above aspects, the amino acid sequence of the non-natural olivetol synthase comprises one or more amino acid variations at position(s) selected from the group consisting of: 125, 126, 185, 187, 189, 190, 204, 208, 209, 210, 211, 249, 250, 257, 259, 331, and 332 of SEQ ID NO:1. In some embodiments, one or more amino acid variations are conservative substitutions at position(s) selected from the group consisting of: 125, 126, 185, 187, 189, 190, 204, 208, 209, 210, 211, 249, 250, 257, 259, 331, and 332 of SEQ ID NO:1.

In some embodiments, the amino acid sequence of the non-natural olivetol synthase comprises one or more amino acid substitution(s) at position(s) selected from the group consisting of: A125G, A125S, A125T, A125C, A125Y, A125H, A125N, A125Q, A125D, A125E, A125K, A125R, A125W, A125F, A125V, S126G, S126A, S126R, S126N, S126D, S126C, S126Q, S126E, S126H, S126I, S126L, S126K, S126M, S126F, S126T, S126W, S126Y, S126V, D185G, D185A, D185S, D185P, D185C, D185T, D185N, D185E, D185H, D185I, D185L, D185K, D185M, D185F, D185W, D185Y, D185V, M187G, M187A, M187S, M187P, M187C, M187T, M187D, M187N, M187E, M187Q, M187H, M187V, M187L, M187I, M187K, M187R, M187F, M187Y, C189R, C189N, C189Q, C189H, C189I, C189L, C189K, C189M, C189F, C189T, L190G, L190A, L190S, L190P, L190C, L190T, L190D, L190N, L190E, L190Q, L190H, L190V, L190M, L190I, L190K, L190R, L190F, L190W, L190Y, G204A, G204C, G204P, G204V, G204L, G204I, G204M, G204F, G204W, G204S, G204T, G204Y, G204H, G204N, G204Q, G204D, G204E, G204K, G204R, F208Y, G209A, G209C, G209P, G209V, G209L, G209I, G209M, G209F, G209W, G209S, G209T, G209Y, G209H, G209N, G209Q, G209D, G209E, G209K, G209R, D210A, D210C, D210P, D210V, D210L, D210I, D210M, D210F, D210W, D210S, D210T, D210Y, D210H, D210N, D210Q, D210E, D210K, D210R, G211A, G211C, G211P, G211V, G211L, G211I, G211M, G211F, G211W, G211S, G211T, G211Y, G211H, G211N, G211Q, G211D, G211E, G211K, G211R, G249A, G249C, G249P, G249V, G249L, G249I, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, L257P, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M331I, M331K, M331R, S332G, and S332A of SEQ ID NO:1. In some embodiments of the above aspects, an olivetol synthase having at least one amino acid substitution as compared to its corresponding natural olivetol synthase, or an olivetol synthase having one or more variations that are different than one or more variations provides improved activity. For example, an olivetol synthase with a different mutation which may have been previously engineered can be used as a template, prior to incorporating any modification described herein. Such olivetol synthases that are starting sequences for incorporating a modification described herein to generate the novel engineered enzyme may be alternatively referred to herein as wild-type, template, starting sequence, natural, naturally-occurring, unmodified, corresponding natural olivetol synthase, corresponding natural olivetol synthase without the amino acid substitution, corresponding olivetol synthase or corresponding olivetol synthase without the amino acid substitution(s). A number of amino acid positions along the length of the olivetol synthase sequence can be substituted to provide non-natural olivetol synthase having increased activity and desired specificity. A single substitution or combinations of substitutions in an olivetol synthase template can provide increased activity and desired specificity, and therefore provide single and combination variants of a starting or template or corresponding olivetol synthase, e.g., in particular enzymes of the class E.C 2.3.1.206, having increased substrate conversion and/or specificity.

### DESCRIPTION OF THE DRAWINGS

FIG. 1 shows an exemplary olivetolic acid synthesis pathway and exemplary cannabigerolic acid synthesis pathway. The terms tetraketide synthase (TKS) and olivetol synthase (OLS) are used interchangeably.

FIG. 2 shows the chemical structures of exemplary acyl-CoA substrate molecules that can be used in an olivetol synthase-catalyzed reaction.

FIG. 3 shows an alignment of SEQ ID NO: 1 (Cannabis sativa BAG14339) to other olivetol synthase and polyketide synthase homologs (SEQ ID NOs: 2-10).

FIG. 4 shows the exemplary pathway for producing olivetolic acid, analogs of olivetolic acid, cannabigerolic acid, analogs of cannabigerolic acid, cannabigerol and analogs of cannabigerol.

FIG. 5 shows the chemical structures of 3,5,7-trioxododecanoyl-CoA, PDAL, Olivetol, HTAL, and olivetolic acid.

FIG. 6A shows exemplary pathways of forming geranyl pyrophosphate from isoprenol, and FIG. 6B shows exemplary pathways of forming geranyl pyrophosphate from geraniol.

FIG. 7 shows exemplary pathways of forming geranyl pyrophosphate from prenol.

FIG. 8 shows exemplary mevalonate pathway (MVA) and non-mevalonate pathway (MEP). The abbreviations are DXS: 1-Deoxy-D-xylulose 5-phosphate synthase; DXR: 1-Deoxy-D-xylulose 5-phosphate reductoisomerase; CMS: 2-C-methyl-D-erythritol 4-phosphate cytidylyltransferase; CMK: 4-diphosphocytidyl-2-C-methyl-D-erythritol kinase; MECS: 2-C-methyl-D-erythritol 2,4-cyclodiphosphate synthase; HDS: 4-Hydroxy-3-methyl-but-2-enyl pyrophosphate synthase; HDR: 4-Hydroxy-3-methyl-but-2-enyl pyrophosphate reductase; DMAP: Dimethylallyl pyrophosphate; AACT: acetoacetyl-CoA thiolase; HMGS: HMG-CoA synthase; HMGR: HMG-CoA reductase; MVK: mevalonate-3-kinase; PMK: Phosphomevalonate kinase; MVD: mevalonate-5-pyrophosphate decarboxylase; and IDI: isopentenyl pyrophosphate isomerase.

FIG. 9 shows the structures of olivetolic acid and exemplary analogs of olivetolic acid.

### DETAILED DESCRIPTION

The embodiments of the description described herein are not intended to be exhaustive or to limit the disclosure to the precise forms disclosed in the following detailed description. Rather, the embodiments are chosen and described so that others skilled in the art can appreciate and understand the principles and practices of the description.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein. Generally, the disclosure provides a non-natural olivetol synthase (OLS) comprising at least one amino acid variation as compared to a wild type olivetol synthase, wherein the non-natural olivetol synthase: a) forms olivetolic acid or olivetol from malonyl-CoA and hexanoyl-CoA at a greater rate as compared to the wild type olivetol synthase; (b) has a higher affinity for hexanoyl-CoA and/or other acyl-CoA substrates as compared to the wild type olivetol synthase; (c) forms olivetolic acid analogs, olivetol analogs, variants thereof, or combinations thereof from malonyl-CoA and other acyl-CoA at a greater rate as compared to the wild type olivetol synthase; (d) is characterized by a lower amount of one or more pyrone-based compounds being formed in the presence of the non-natural olivetol synthase (OLS) as compared to the wild type olivetol synthase, or (e) any combination of (a), (b), (c) or (d), wherein olivetolic acid or olivetol, analogs thereof, variants thereof, or acid derivatives of a polyketide are formed in the presence of olivetolic acid cyclase (OAC) not rate limited by amount of activity.

Olivetol synthase (OLS) belongs to plant type III polyketide synthases (PKS) which are a group of condensing enzymes that catalyze the initial key reactions in the biosynthesis of a myriad of secondary metabolites. All the plant type III polyketide synthases that have been characterized are homodimeric proteins. Each monomer of the dimeric protein contains its own active site and catalyzes the sequential condensation of starter CoA molecule and one acyl unit from malonyl-CoA, independently. Each condensation step is associated with one decarboxylation step.

Structure-function analyses of plant PKSs have suggested that numerous biosynthetic enzymes including olivetol synthase are evolved from chalcone synthase, the ubiquitous plant type III PKS catalyzing the first committed step in flavonoid biosynthesis, by changing active site residues regulating substrate specificity and/or cyclization reactions of linear polyketide intermediates (Austin & Noel, Nat. Prod. Rep., 20:79-110). For example, crystal structure analyses of chalcone synthase (CHS) and stilbene synthase (STS) have suggested that only a small number of amino acid substitutions in CHS alter the cyclization reaction from Claisen-type into aldol-type, and that STS evolved from CHS with this functional change called the aldol switch (Ferrer et al., Nat. Struct. Biol., 6:775-784; Austin et al., Chem. Biol., 11:1179-1194).

Olivetol synthases are classified as EC:2.3.1.206 under the Enzyme Commission nomenclature. Olivetol synthases have structural similarities with plant type III PKS enzymes. The OLS enzyme comprises conserved Cys157-His 297-Asn 330 catalytic triad, and the ‘gatekeeper’ Phe 208 corresponding to the amino acid positions of SEQ ID NO: 1. These amino acid residues are conserved for all other OLS homologs corresponding to SEQ ID NOs: 2-10.

SEQ ID NOs: 1-10 have the following identities. SEQ ID NO:1: 3,5,7 trioxododecanoyl-CoA synthase (OLS) from Cannabis sativa, 385 aa, Accession Number BAG14339/B1Q2B6; SEQ ID NO:2: Polyketide synthase 3 (PKSG3) from Cannabis sativa, 385 aa, Accession Number F1LKH5 (99.5% identity to SEQ ID NO:1); SEQ ID NO:3: Polyketide synthase 1 (PKSG1) from Cannabis sativa, 385 aa, Accession Number F1LKH6 (98.4% identity to SEQ ID NO:1); SEQ ID NO:4: Polyketide synthase 2 (PKSG2) from Cannabis sativa, 385 aa, Accession Number F1LKH7 (97.7% identity to SEQ ID NO:1); SEQ ID NO:5: Polyketide synthase 4 (PKSG4) from Cannabis sativa, 385 aa, Accession Number F1LKH8 (98.7% identity to SEQ ID NO:1); SEQ ID NO:6: Polyketide synthase 5 (PKSGS) from Cannabis sativa, 385 aa, Accession Number F1LKH9 (98.2% identity to SEQ ID NO:1); SEQ ID NO:7: Coumaroyl triacetic acid synthase from Hydrangea macrophylla (HmCTAS), 399 aa, Accession Number BAA32733.1 (57.5% identity to SEQ ID NO:1); SEQ ID NO:8: Stilbenecarboxylate synthase from Hydrangea macrophylla (HmSCTS1), 399 aa, Accession Number AAN76182.1 (57.7% identity to SEQ ID NO:1); SEQ ID NO:9: Stilbenecarboxylate synthase from Hydrangea macrophylla (HmSCTS2), 399 aa, Accession Number AAN76183.1 (57.5% identity to SEQ ID NO:1); SEQ ID NO:10: Stilbenecarboxylate synthase 2 from Marchantia polymorpha (MpSCTS2), and 392 aa, Accession Number AAW30010.1 (52.7% identity to SEQ ID NO:1). (BLAST parameters: EBLOSUM62; Gap_penalty: 10.0; Extend_penalty: 0.5.)

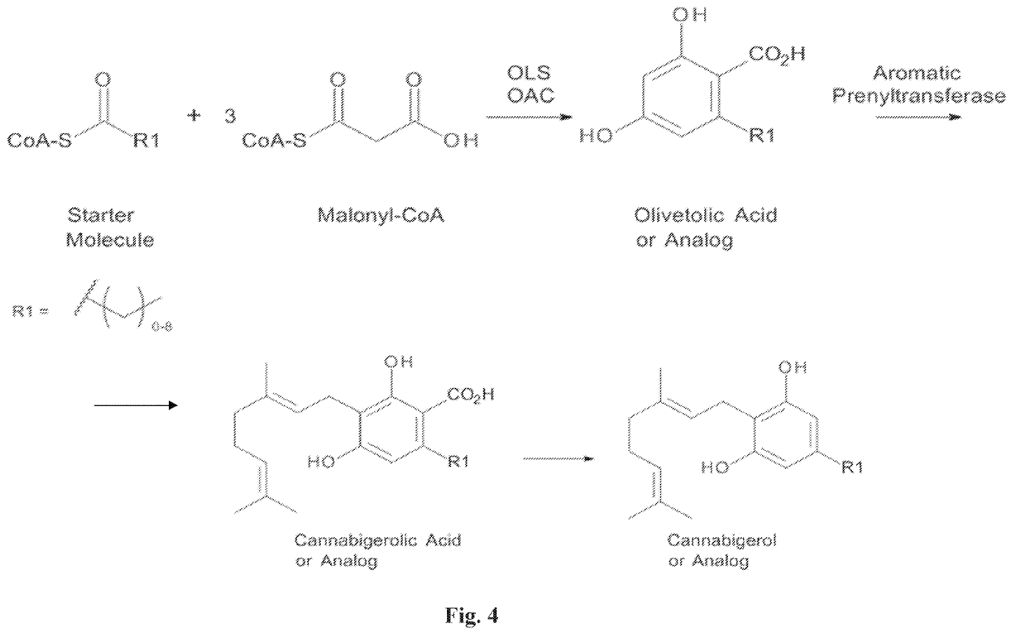

As used herein, an “analog” (alternatively referred to as a “structural analog” or “chemical analog”) of compounds of the disclosure refers to a compound having a structure that is similar to that of another compound, but that differs from the compound with respect to a certain aspect of the compound, such as a chemical group. Analogs include “substrate analogs”, such as structurally-related chemical compounds that can be used by a common enzyme (e.g., OLS). Examples of analogs include acyl-coA compounds, wherein propionyl-CoA, butyryl-CoA, and valeryl-CoA, etc., are examples of analogs of acetyl-CoA. As another example, and with reference to FIG. 4, analogs of cannabigerolic acid (CBGA) include those compounds having the base [(2E)-3,7-dimethylocta-2,6-dien-1-yl]-2,4-dihydroxybenzoic acid structure, but with different R¹ chemical groups (e.g., 3-[(2E)-3,7-dimethylocta-2,6-dien-1-yl]-2,4-dihydroxy-6-propylbenzoic acid and 3-[(2E)-3,7-dimethylocta-2,6-dien-1-yl]-2,4-dihydroxy-6-butylbenzoic acid are analogs of (3-[(2E)-3,7-dimethylocta-2,6-dien-1-yl]-2,4-dihydroxy-6-pentylbenzoic acid (CBGA)).

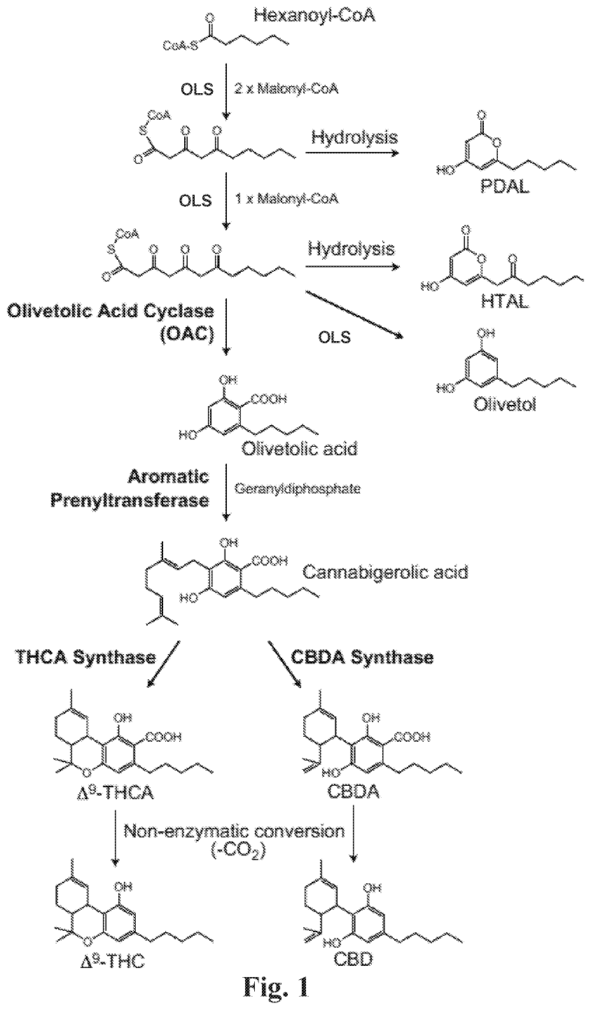

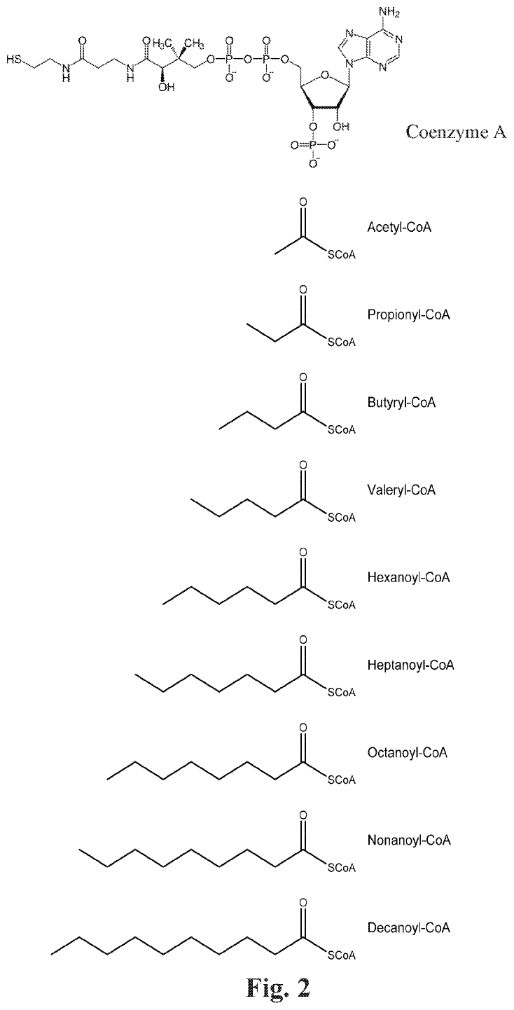

As used herein, a “derivative” (alternatively referred to as a “chemical derivative”) of compounds of the disclosure refers to a compound or compounds chemically derived from a precursor chemical compound. As an example, and with reference to FIG. 1, 3,5,7-trioxododecanoyl-CoA is a derivative of hexanoyl-CoA, cannabigerolic acid (CBGA) is a derivative of olivetolic acid (OA), and CBDA is a derivative of CBGA.

As used herein, polyketides refer to compounds containing alternating carbonyl and methylene groups (—CO—CH₂—) and are also known as “β-polyketones”. Polyketides can include compounds derived from repeated decarboxylative condensation of malonyl coenzyme A.

An exemplary polyketide generated by OLS is 3,5,7-trioxododecanoyl-CoA. The 3,5,7-trioxododecanoyl-CoA, a linear polyketide, has the following chemical names: 3,5,7-trioxododecanoyl-coenzyme A; 3,5,7-trioxolauroyl-CoA; 3,5,7-trioxolauroyl-coenzyme A; and 3′-phosphoadenosine 5′-(3-{(3R)-3-hydroxy-2,2-dimethyl-4-oxo-4-[(3-oxo-3- {[2-(3,5,7-trioxododecanoylsulfanyl)ethyl]amino}propyl)amino]butyl} dihydrogen diphosphate). In some embodiments, the non-naturally occurring olivetol synthase (OLS) preferentially catalyzes the condensation of malonyl-CoA and acyl-CoA (non-limiting examples include acetyl-CoA, propionyl-CoA, butyryl-CoA, valeryl-CoA, hexanoyl-CoA, heptanoyl-CoA, octanoyl-CoA, nonanoyl-CoA, decanoyl-CoA) to form polyketides such as 3,5,7-trioxododecanoyl-CoA and 3,5,7-trioxododecanoate and their analogs. The polyketides can be converted to olivetolic acid and its analogs in the presence of olivetolic acid cyclase (OAC) enzyme.

Olivetol may also be formed from a polyketide intermediate. In the absence of OAC, and in the presence of a non-limiting supply of malonyl-CoA, the OLS can convert the polyketides into olivetol or its analogs (see FIG. 1), olivetol being a predominant product. Olivetol is also known by the chemical names 5-pentylbenzene-1,3-diol, 5-pentylresorcinol, and 5-pentyl-1,3-benzenediol. In the absence of olivetolic acid cyclase (OAC), olivetol can be formed as an OLS-catalyzed resorcinol (1,3 -dihydroxybenzene)-containing product.

However, there is a competing reaction where polyketide substrate(s) are hydrolyzed to pyrone compounds, such as lactones like pentyl diacetic acid lactone (PDAL), hexanoyl triacetic acid lactone (HTAL), and other pyrone analogs depending on the starting substrates. PDAL, a pyrone by-product of olivetol synthase-catalyzed reaction caused by hydrolysis of the polyketide substrate, has the chemical name pentyl diacetic acid lactone. HTAL, another hydrolysis pyrone by-product of OLS-catalyzed reaction formed by hydrolysis of 3,5,7-trioxododecanoyl-CoA has the chemical name hexanoyl triacetic acid lactone. In embodiments of the disclosure pyrone-containing compounds such as PDAL and HTAL can be considered “derailment products” or “byproducts” and may be formed from polyketide intermediates. Tetraketide and triketide pyrones were reported to be the reaction products of various type III PKSs, and triketide pyrone could be a derailment product from a premature intermediate. In embodiments of the disclosure, in the presence of the non-natural olivetol synthase (OLS) lower amounts of pyrone-containing compounds such as PDAL and HTAL relative to wild type OLS are formed. Accordingly, the lower amounts can be observed as shift in the ratio of a desired compound(s) (e.g., olivetol, olivetolic acid, or analogs or derivatives thereof) to the pyrone-containing compound(s) (e.g., PDAL, HTAL, or analogs or derivatives thereof) relative to the non-natural olivetol synthase (OLS). The formation of olivetol over pyrone byproducts such as PDAL and/or HTAL can be measured in the presence of the non-natural OLS but the absence of OAC, which otherwise converts the polyketide intermediate to olivetolic acid.

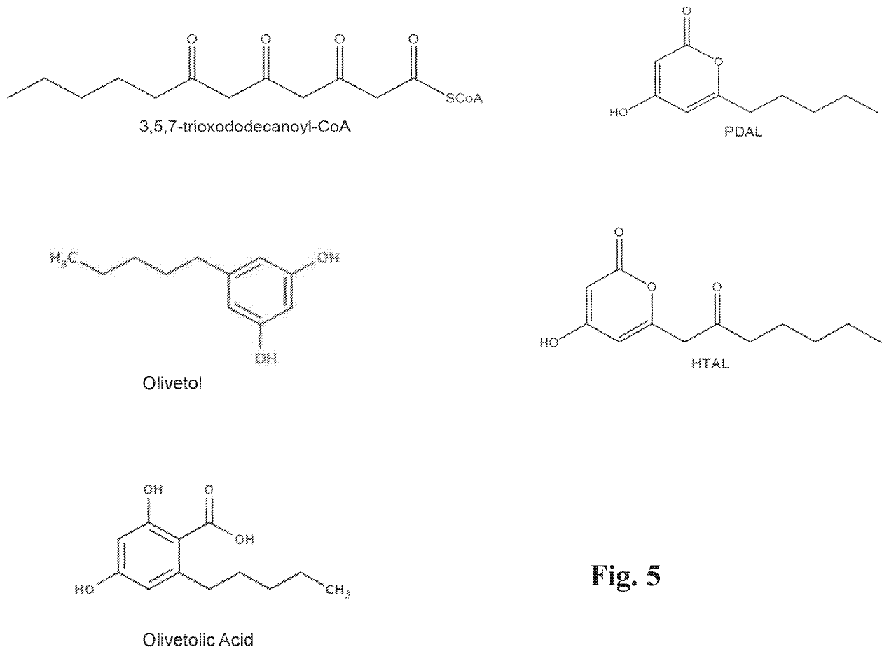

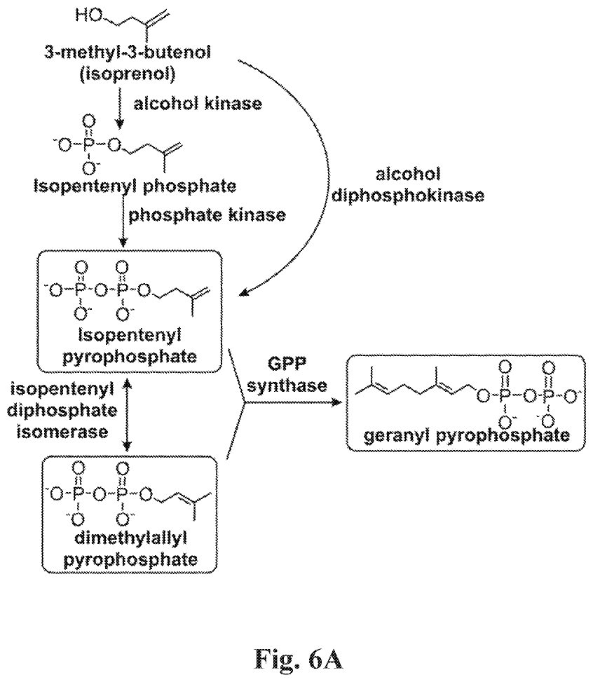

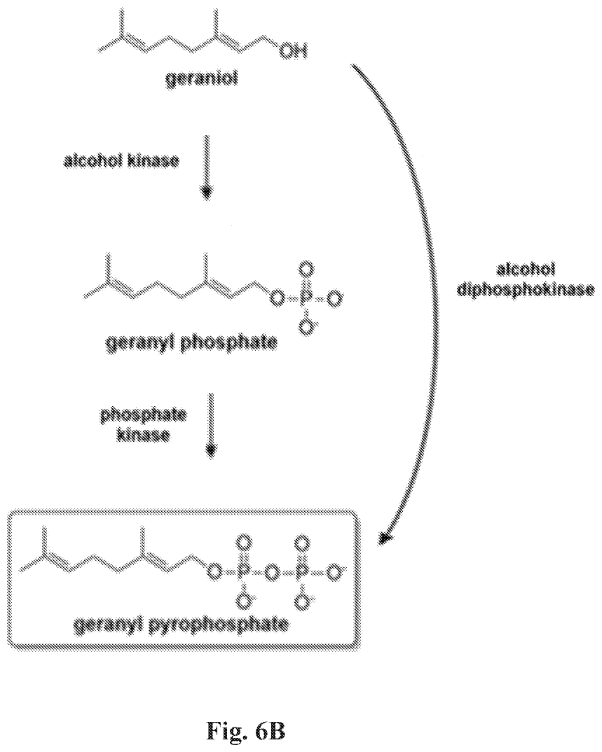

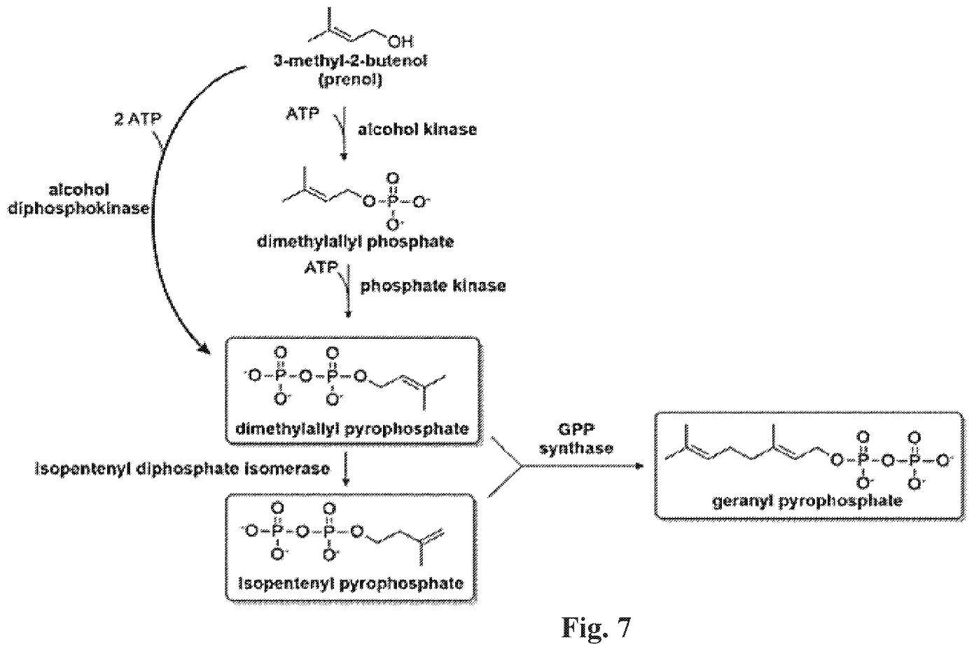

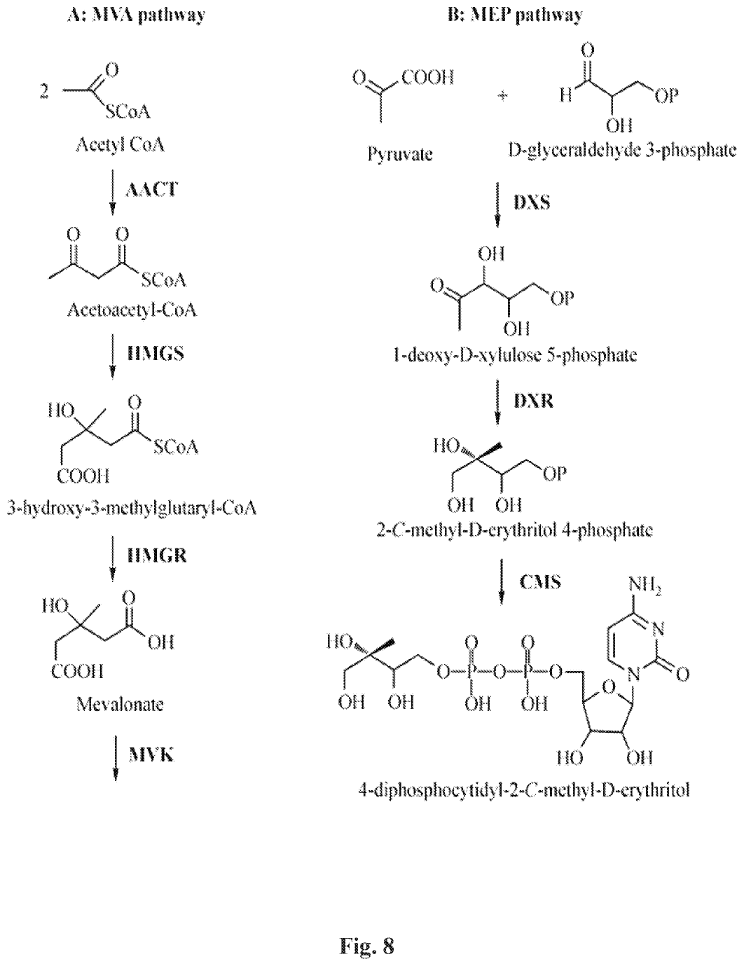

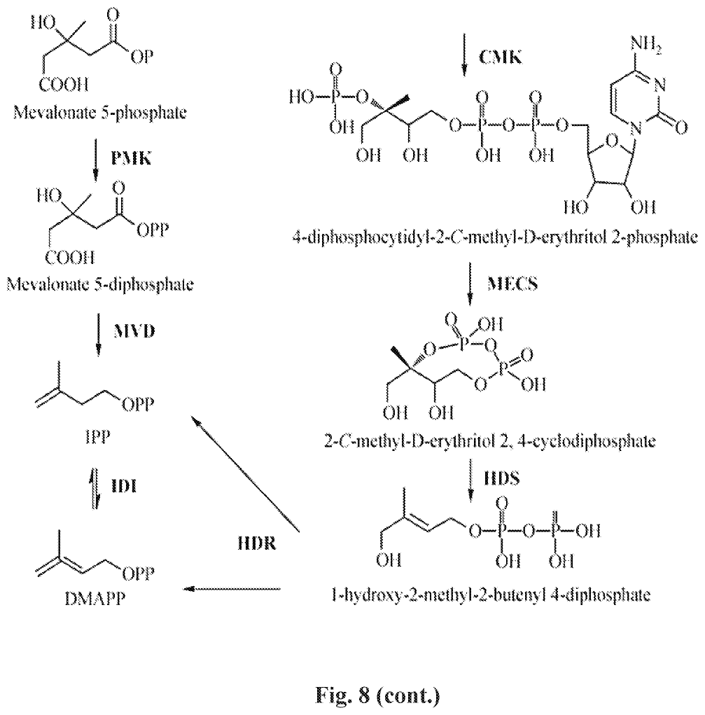

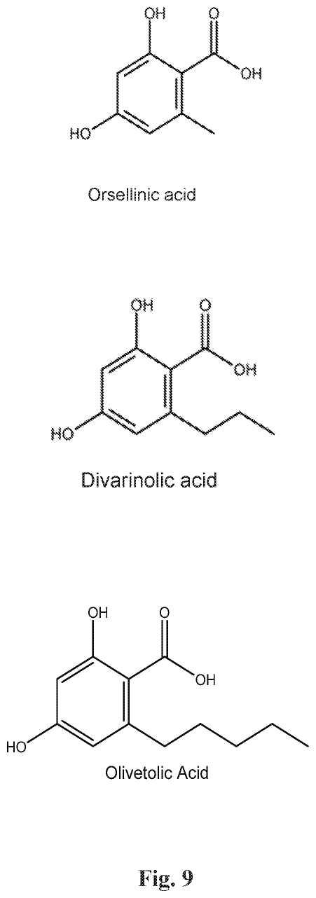

Olivetolic acid (OLA) can also be chemically referred to as olivetolate, 2,4-dihydroxy-6-pentylbenzoic acid, or olivetol-6-carboxylic acid. The chemical structures of 3,5,7-trioxododecanoyl-CoA, olivetol, OLA, PDAL, and HTAL are shown in FIG. 5.

In some embodiments, the amino acid sequence of the non-natural olivetol synthase has at least about: 50%, 60%, 65%, 70%, 75%, 80%, 85%, 87.5%, 90%, 92.5%, 93%, 94%, 95%, 96%, 97%, 98%, 99%, or 100% sequence identity to at least 10, 25, 30, 35, 40, 50, 55, 60, 70, 75, 80, 90, 95, 100, 110, 120, 130, 140, 150, 160, 170, 180, 190, 200, 250, 300, 350, 355, 360, 365, 370, 375, 385, or more, or all, contiguous amino acids of any one of the amino acid sequences of SEQ ID NOs:1-10. As used herein, “at least about 50%,” “at least about 60%,” etc., is the same as about 50% or greater, about 60% or greater, etc., respectively.

An amino acid “variation” (herein “variation” and “mutation” can be used interchangeably) is a change of an amino acid at a particular position in the referenced olivetol synthase template to a variant amino acid at that position.

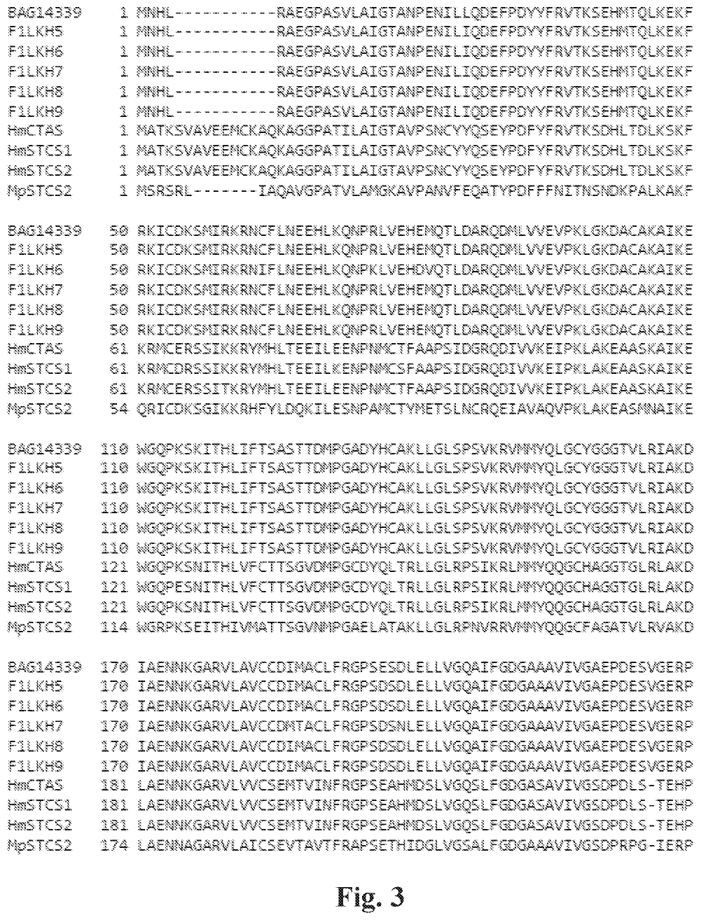

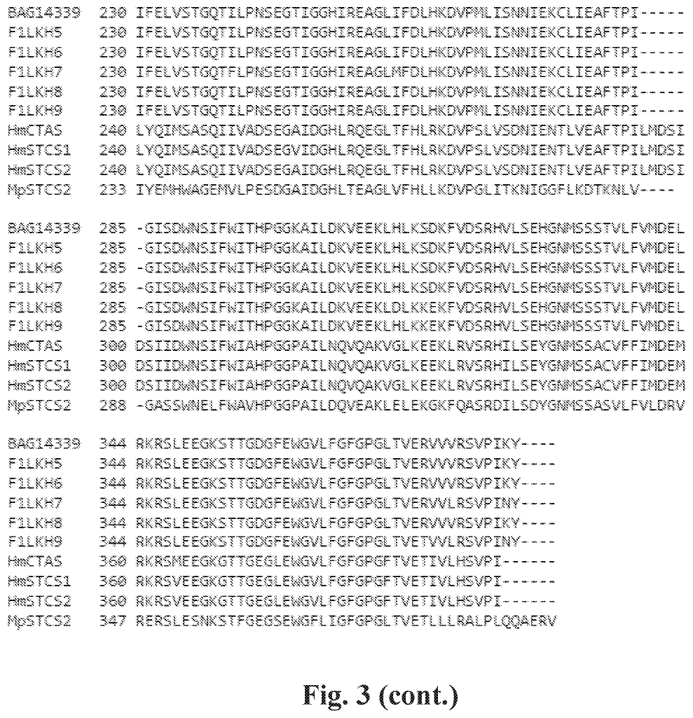

In some embodiments, the amino acid sequence of the non-natural olivetol synthase has one or more amino acid variations at position(s) selected from the group consisting of: 82, 125, 126, 131, 185, 186, 187, 189, 190, 195, 197, 204, 208, 209, 210, 211, 249, 250, 257, 259, 314, 331, and 332 corresponding to the amino acid sequence of SEQ ID NO:1. Although the positions recited herein are with reference to the corresponding amino acid sequence of SEQ ID NO:1, it is expressly contemplated that the amino acid sequence of the non-natural olivetol synthase can have one or more amino acid variations at equivalent positions (variant positions) corresponding to the homologs of SEQ ID NO: 1, e.g., SEQ ID NOs: 2-10. As shown in FIG. 3, SEQ ID NOs 1-10 align very well and therefore identification of variant positions in any of SEQ ID NOs: 2-10 that correspond to variant positions in SEQ ID NO:1 can readily be understood.

For example, in SEQ ID NO:7 the variant positions are shifted +10-15, from these locations, and therefore SEQ ID NO:7 can have one or more amino acid variations at position(s) selected from the group consisting of: 93, 136, 137, 142, 196, 197, 198, 200, 201, 206, 208, 215, 219, 220, 221, 222, 259, 260, 267, 269, 329, 346, and 347 with reference to the amino acid sequence of SEQ ID NO:1. As another example, in SEQ ID NO:10 the variant positions are shifted by +3-4, from these locations, and therefore SEQ ID NO:10 can have one or more amino acid variations at position(s) selected from the group consisting of: 86, 129, 130, 135, 189, 190, 191, 193, 194, 199, 201, 208, 212, 213, 214, 215, 252, 253, 260, 262, 317, 334, and 335 with reference to the amino acid sequence of SEQ ID NO:1. Further, other olivetol synthases that are different than SEQ ID NOs: 1-10 can be aligned to SEQ ID NO: 1 to identify variant positions and used to create non-natural olivetol synthases that are different than non-natural olivetol synthases based on SEQ ID NOs: 1-10 of the disclosure. In some embodiments, other olivetol synthases that are different than SEQ ID NOs 1-10, but having amino acid identity of 50% or greater, can be aligned to SEQ ID NO: 1 to identify corresponding variant amino acid positions and to make non-natural olivetol synthases based on information of the current disclosure.

In some embodiments, the amino acid substitutions designed to increase olivetolic acid production by OLS are shown below. The amino acid positions of OLS corresponds to SEQ ID NO: 1. It is expressly contemplated that the amino acid sequence of the non-natural olivetol synthase can have one or more amino acid variations at equivalent positions corresponding to the homologs of SEQ ID NO: 1, e.g., SEQ ID Nos 2-10 (Table 1).

TABLE 1

| Position | Substitution |
|---|---|
| A125 | G,S,T,C,Y,H,N,Q,D,E,K,R,W,F,V |
| S126 | G,A,R,N,D,C,Q,E,H,I,L,K,M,F,T,W,Y,V |
| D185 | G,A,S,P,C,T,N,Q,E,J,I,L,K,M,F,W,Y,V,H |
| M187 | G,A,S,P,C,T,D,N,E,Q,H,V,L,I,K,R,F,Y |
| C189 | R,N,Q,H,I,L,K,M,F,T |
| L190 | G,A,S,P,C,T,D,N,E,Q,H,V,M,I,K,R,F,W,Y |
| G204 | A,C,P,V,L,I,M,F,W |
| F208 | Y |
| G209 | A,C,P,V |
| D210 | A,C,P,V |
| G211 | A,C,P,V |
| G249 | A,C,P,V,L,I,M,F,W,S,T,Y,H,N,Q,D,E,K,R |
| G250 | A,C,P,V,L,I,M,F,W,S,T,Y,H,N,Q,D,E,K,R |
| L257 | V,M,I,K,R,F,Y,W,S,T,C,H,N,Q,D,E,P |
| F259 | G,A,C,P,V,L,I,M,Y,W,S,T,Y,H,N,Q,D,E,K,R |
| M331 | G,A,S,P,C,T,D,N,E,Q,H,V,L,I,K,R |
| S332 | G,A |

For example, in some embodiments, in a non-natural olivetol synthase of the disclosure based on any one of SEQ ID NOs: 1-5, there can be an amino acid variant selected from G, S, T, C, Y, H, N, Q, D, E, K, W, F, V, or R at position 125, which replaces the wild type A. However, the corresponding position in SEQ ID NO:7 is shifted +11, which corresponds to position 136. Since the wild type amino acid at position 136 in SEQ ID NO:7 is already T, the amino acid variant can be selected from G, S, C, Y, H, N, Q, D, E, K, W F, V, and R (i.e., excluding the wild-type T as a possibility) for position 136 to create a non-natural olivetol synthase. In embodiments wherein a single amino acid variant is prescribed at a certain amino acid position, but the prescribed substitution is already present as a wild type amino acid at that position, then another variant amino acid position is looked to so the non-natural olivetol synthase can be based on a non-wild type, prescribed variant, amino acid.

In some embodiments the non-natural olivetol synthase comprises one or more amino acid substitutions at position(s) selected from the group consisting of: Q82S, P131A, I186F, M187E, M187N, M187T, M187I, M187S, M187A, M187L, M187G, M187V, M187C, S195K, S195M, S195R, S197G, S197V, K314D, and K314M, corresponding to the amino acid positions of SEQ ID NO:1. One or more of the recited substitutions can be made in SEQ ID NO:1, an olivetol synthase having sequence identity to SEQ ID NO:1 (e.g., at least about 50%, 75%, 90%, 93%, 94%, 95%, 96%, 97%, 98%, 99% identity, etc.), or at one or more corresponding amino acid locations in any of SEQ ID NOs:2-10 or an olivetol synthase having sequence identity to any of SEQ ID NOs:2-10 (e.g., at least about 50%, 75%, 90%, 93%, 94%, 95%, 96%, 97%, 98%, 99% identity, etc.).

In some embodiments the non-natural olivetol synthase comprises two or more amino acid substitutions, wherein one or more substitution(s) is/are at position(s) selected from the group consisting of: Q82S, P131A, I186F, M187E, M187N, M187T, M187I, M187S, M187A, M187L, M187G, M187V, M187C, S195K, S195M, S195R, S197G, S197V, K314D, and K314M, and one or more of another amino acid substitutions is at position(s) selected from the group consisting of amino acid substitutions described in Table 1 herein. A non-natural olivetol synthase comprising these two or more amino acid substitutions can be made in in SEQ ID NO:1, an olivetol synthase having sequence identity to SEQ ID NO:1 (e.g., at least about 50%, 75%, 90%, 93%, 94%; 95%, 96%, 97%, 98%, 99% identity, etc.), or at one or more corresponding amino acid locations in any of SEQ ID NOs:2-10 or an olivetol synthase having sequence identity to any of SEQ ID NOs: 2-10 (e.g., at least about 50%, 75%, 90%, 93%, 94%, 95%, 96%, 97%, 98%, 99% identity, etc.)

In some embodiments, non-natural olivetol synthase with one or more variant amino acids as describe herein are enzymatically capable of preferentially forming polyketides as opposed to PDAL, HTAL, or other pyrone analogs as compared to the wild-type enzyme. The polyketides can be hydrolyzed to PDAL, HTAL, and other pyrone analogs depending on the starting substrates, or the polyketides can be converted to olivetol and its analogs by olivetol synthase.

The polyketides also work as substrates for olivetolic acid cyclase, which converts the polyketides to olivetolic acid and its analogs depending on the starting substrates.

In some embodiments, non-natural olivetol synthase with one or more variant amino acids as described herein are enzymatically capable of at least about 1.2, 1.5, 2, 3, 4, 5, 6, 7, 8, 9, 10, 12, 15, 20, or greater rate of formation of olivetolic acid from malonyl-CoA and hexanoyl-CoA in the presence of olivetolic acid cyclase (OAC) enzyme not rate limited by amount or activity, or rate of formation of olivetol without OAC, as compared to the wild type olivetol synthase.

In some embodiments wherein olivetolic acid, an analog thereof, or derivative thereof, is formed, the OAC is present in molar excess of OLS. In some embodiments, the molar ratio of OLS to OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more.

For example, in the presence of OAC not rate limited by amount or activity there is an increase in rate of formation of olivetolic acid from malonyl-CoA and hexanoyl-CoA, or alternatively, in the absence of OAC there is an increase in rate of formation of olivetol from malonyl-CoA and hexanoyl-CoA, as compared to the wild olivetol synthase, that is about at least 1.01-times greater as compared to the rate with wild type olivetol synthase. In some embodiments the rate of olivetolic acid or olivetol formation using the non-natural olivetol synthase is at least about 1.02 times, about 1.03 times, about 1.04 times, about 1.05 times, about 1.06 times, about 1.07 times, about 1.08 times, about 1.09 times, about 1.1 times, about 1.12 times, about 1.14 times, about 1.16 times, about 1.18 times, about 1.2 times, about 1.24 times, about 1.28 times, about 1.32 times, about 1.36 times, or about 2-times greater as compared to the rate with wild type olivetol synthase as determined in an in vitro enzymatic reaction using purified olivetol synthase variant. In some embodiments the rate of olivetolic acid or olivetol formation using the non-natural olivetol synthase is in the range of greater than 1.01 times to about 300 times, about 1.02 times to about 2 times, about 1.2 times to about 300 times, about 1.5 times to about 200 times, or about 2 times to about 30 times as determined in an in vitro enzymatic reaction using purified olivetol synthase variant.

Formation of one or more non-target products relative to one or more target products can also be minimized in the presence of the non-natural OLS. For example, in some embodiments, the total non-target products (e.g., by-products such as PDAL, HTAL, and other pyrone analogs) are in an amount (w/w) of less than about 50%, 45%, 40%, 35%, 30%, 25%, 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by OLS and OAC enzyme combinations.

Lower amounts of non-target products, such as pyrone-containing compounds like PDAL and HTAL, can be formed in the presence of a non-natural olivetol synthase (OLS) that relative to wild type OLS. The lower amount of pyrone-containing compounds can be observed as shift in the ratio of olivetol or olivetolic acid to PDAL and/or HTAL in the presence of the non-natural olivetol synthase (OLS) relative to the wild type OLS. Accordingly, in some embodiments, in the presence of a non-natural olivetol synthase there is a target product (e.g. olivetol, olivetolid acid):byproduct (PDAL, HTAL) ratio (mol) that is greater than the target product:byproduct ratio (mol) in the presence of the wild type olivetol synthase. The target product can be a polyketide or alcohol or acid derivative thereof, and the byproduct can be a pyrone-based hydrolysis product of the polyketide or derivative thereof. Using an in vitro enzymatic reaction, the target product to byproduct ratio can be determined in the presence of OAC (not rate limited by amount or enzymatic form), or can be determined without OAC. For example, in the presence of a non-rate limiting amount of OAC, a target product such as olivetolic acid (OLA) can be formed, and can be compared to a by-product such as pentyl diacetic acid lactone (PDAL). Alternatively, without OAC, olivetol (OL) can be formed as a representative “target product”, and can be compared to a by-product such as pentyl diacetic acid lactone (PDAL).

In some aspects the target product:byproduct ratio (mol) formed in the presence of the non-natural olivetol synthases is about 1.1-fold or greater than the target product:byproduct ratio (mol) formed in the presence of the wild type olivetol synthase. In more specific embodiments, in the presence of the non-natural olivetol synthase a target product:byproduct ratio (mol) is formed that is about 1.2-fold, about 1.3-fold, about 1.4-fold, about 1.5-fold, about 1.6-fold, about 1.8-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, or about 3.0-fold or greater than a target product:byproduct ratio (mol) formed by the wild type olivetol synthase.

In some embodiments the non-natural olivetol synthase comprises one amino acid substitution, or more than amino acid substitutions, at a position selected from the group consisting of: Q82S, P131A, I186F, M187S, S195K, S195M, S197V, T293E, K314D, and K314M, corresponding to the amino acid positions of SEQ ID NO:1. The non-natural olivetol synthase can a) forming olivetolic acid or olivetol from malonyl-CoA and hexanoyl-CoA at a greater rate as compared to the wild type olivetol synthase and/or can form one or more pyrone-based hydrolysis product(s) at a rate that is less than the wild type olivetol synthase. The one or more of the recited substitutions can be made in SEQ ID NO:1, an olivetol synthase having sequence identity to SEQ ID NO:1 (e.g., at least about 50%, 75%, 90%, 93%, 94%, 95%, 96%, 97%, 98%, 99% identity, etc.), or at one or more corresponding amino acid locations in any of SEQ ID NOs: 2-10 or an olivetol synthase having sequence identity to any of SEQ ID NOs: 2-10 (e.g., at least about 50%, 75%, 90%, 93%, 94%, 95%, 96%, 97%, 98%, 99% identity, etc.).

In some embodiments the non-natural olivetol synthase comprises two, or more than two amino acid substitutions, with at least one (i.e., the first) amino acid substitution at a position selected from the group consisting of: Q82S, P131A, I186F, M187S, S195K, S195M, S197V, T293E, K314D, and K314M, corresponding to the amino acid positions of SEQ ID NO:1. In some embodiments, the second amino acid substitution is at a position selected from the group consisting of Q82S, P131A, I186F, M187S, S195K, S195M, S197V, T293E, K314D, and K314M.

In embodiments non-natural olivetol synthase comprises two, or more than two amino acid substitutions, selected from: (i) Q82S and P131A, (ii) Q82S and M187S, (iii) Q82S and S195K, (iv) Q82S and S195M, (v) Q82S and S197V, (vi) Q82S and K314D, (vii) P131A and I186F, (viii) P131A and M187S, (ix) P131A and S195M, (x) P131A and S197V, (xi) P131A and K314D, (xii) P131A and K314M, (xiii) I186F and M187S, (xiv) I186F and S195K, (xv) I186F and S195M, (xvi) I186F and T239E, (xvii) I186F and K314D, (xviii) M187S and S195K, (xix) M187S and S195M, (xx) M187S and S197V, (xxi) M187S and T239E, (xxii) M187S and K314D, (xxiii) M187S and K314M, (xxiv) S195K and S197V, (xxv) S195M and S197V, (xxvi) S195M and T239E, (xxvii) S195K and K314D, (xxviii) S195K and K314M, (xxix) S195M and K314D, (xxx) S195M and K314M, (xxxi) S197V and T239E, (xxxii) S197V and K314M, (xxxiii) T239E and K314D, (xxxiv) T239E and K314M, (xxxv) Q82S and I186F, (xxxvi) Q82S and T239E, (xxxvii) Q82S and K314M, (xxxviii) I186F and S197V (xxxix) I186F and K314M, (xl) S195K and T239E, (xli) S197V and K314D, (xlii) P131A and T239E, and (xliii) P131A and S195K. The two or more of the recited substitutions of any of (i) to (xliii) can be made in SEQ ID NO:1, an olivetol synthase having sequence identity to SEQ ID NO:1 (e.g., at least about 50%, 75%, 90%, 93%, 94%, 95%, 96%, 97%, 98%, 99% identity, etc.), or at two or more corresponding amino acid locations in any of SEQ ID NOs:2-10 or an olivetol synthase having sequence identity to any of SEQ ID NOs:2-10 (e.g., at least about 50%, 75%, 90%, 93%, 94%, 95%, 96%, 97%, 98%, 99% identity, etc.). Non-natural olivetol synthases having two of substitutions (i) to (xliii) include those that are capable of forming olivetolic acid or olivetol from malonyl-CoA and hexanoyl-CoA at a greater rate as compared to the wild type olivetol synthase and/or can form one or more pyrone-based hydrolysis product(s) at a rate that is less than the wild type olivetol synthase.

In embodiments non-natural olivetol synthase comprises three, or more than three, amino acid substitutions selected from: (i) Q82S, P131A, and I186F, (ii) Q82S, P131A, and M187S, (iii) Q82S, P131A, and S195K, (iv) Q82S, P131A, and S195M, (v) Q82S, P131A, and S197V, (vi) Q82S, P131A, and T239E, (vii) Q82S, P131A, and K314D, (viii) Q82S, P131A, and K314M, (ix) Q82S, I186F, and M187S, (x) Q82S, I186F, and S195M, (xi) Q82S, I186F, and S197V, (xii) Q82S, I186F, and T239E, (xiii) Q82S, I186F, and K314D, (xiv) Q82S, I186F, and K314M, (xv) Q82S, M187S, and S195K, (xvi) Q82S, M187S, and S195M, (xvii) Q82S, M187S, and S197V, (xviii) Q82S, M187S, and T239E, (xix) Q82S, M187S, and K314D, (xx) Q82S, M187S, and K314M, (xxi) Q82S, S195K, and S197V, (xxii) Q82S, S195M, and S197V, (xxiii) Q82S, S195K, and K314D, (xxiv) Q82S, S195K, and K314M, (xxv) Q82S, S195M, and K314D, (xxvi) Q82S, S195M, and K314M, (xxvii) Q82S, S197V, and T239E, (xxviii) Q82S, S197V, and K314D, (xxix) Q82S, S197V, and K314M, (xxx) Q82S, T239E, and K314D, (xxxi) Q82S, T239E, and K314M, (xxxii) P131A, I186F, and M187S, (xxxiii) P131A, I186F, and S195K, (xxxiv) P131A, I186F, and S195M, (xxxv) P131A, I186F, and S197V, (xxxvi) P131A, I186F, and K314D, (xxxvii) P131A, I186F, and K314M, (xxxviii) P131A, M187S, and S195K, (xxxix) P131A, M187S, and S195M, (xl) P131A, M187S, and S197V, (xli) P131A, M187S, and T239E, (xlii) P131A, M187S, and K314D, (xliii) P131A, S195M, and S197V, (xliv) P131A, S195M, and T239E, (xlv) P131A, S195K, and K314D, (xlvi) P131A, S195K, and K314M, (xlvii) P131A, S195M, and K314D, (xlviii) P131A, S195M, and K314M, (xlix) P131A, S197V, and T239E, (l) P131A, S197V, and K314D, (li) P131A, S197V, and K314M, (lii) P131A, T239E, and K314D, (liii) P131A, T239E, and K314M, (liv) I186F, M187S, and S195K, (lv) I186F, M187S, and S195M, (lvi) I186F, M187S, and S197V, (lvii) I186F, M187S, and K314M, (lviii) I186F, S195K, and S197V, (lix) I186F, S195M, and S197V, (lx) I186F, S195K, and T239E, (lxi) I186F, S195M, and T239E, (lxii) I186F, S195K, and K314D, (lxiii) I186F, S195K, and K314M, (lxiv) I186F, S195M, and K314D, (lxv) I186F, S195M, and K314M, (lxvi) I186F, S197V, and T239E, (lxvii) I186F, S197V, and K314D, (lxviii) I186F, S197V, and K314M, (lxix) I186F, T239E, and K314M, (lxx) M187S, S195K, and S197V, (lxxi) M187S, S195M, and S197V, (lxxii) M187S, S195K, and T239E, (lxxiii) M187S, S195M, and T239E, (lxxiv) M187S, S195K, and K314D, (lxxv) M187S, S195K, and K314M, (lxxvi) M187S, S195M, and K314D, (lxxvii) M187S, S195M, and K314M, (lxxviii) M187S, S197V, and T239E, (lxxix) M187S, S197V, and K314D, (lxxx) M187S, S197V, and K314M, (lxxxi) M187S, T239E, and K314D, (lxxxii) M187S, T239E, and K314M, (lxxxiii) S195K, S197V, and T239E, (lxxxiv) S195M, S197V, and T239E, (lxxxv) S195K, S197V, and K314D, (lxxxvi) S195K, S197V, and K314M, (lxxxvii) S195M, S197V, and K314D, (lxxxviii) S195M, S197V, and K314M, (lxxxix) S195K, T239E, and K314D, (xc) S195K, T239E, and K314M, (xci) S195M, T239E, and K314D, (xcii) S195M, T239E, and K314M, and (xciii) S197V, T239E, and K314M. The three or more of the recited substitutions of any of (i) to (xciii) can be made in SEQ ID NO:1, an olivetol synthase having sequence identity to SEQ ID NO:1 (e.g., at least about 50%, 75%, 90%, 93%, 94%, 95%, 96%, 97%, 98%, 99% identity, etc.), or at three or more corresponding amino acid locations in any of SEQ ID NOs: 2-10 or an olivetol synthase having sequence identity to any of SEQ ID NOs: 2-10 (e.g., at least about 50%, 75%, 90%, 93%, 94%, 95%, 96%, 97%, 98%, 99% identity, etc.). Non-natural olivetol synthases having three of substitutions (i) to (xciii) include those that are capable of forming olivetolic acid or olivetol from malonyl-CoA and hexanoyl-CoA at a greater rate as compared to the wild type olivetol synthase and/or one or more pyrone-based hydrolysis product(s) is formed in an amount that is less than the wild type olivetol synthase.

In some embodiments, the amino acid substitutions designed to alter the starter molecule specificity of the OLS enzyme is shown below. Starter molecule specificity refers to the initial substrate that binds in the active site and is elongated by the addition of extender molecules. For olivetolic acid or olivetol, hexanoyl-CoA is the starter molecule and three malonyl-CoA are the extender molecules. The amino acid positions of OLS corresponds to SEQ ID NO: 1. It is expressly contemplated that the amino acid sequence of the non-natural olivetol synthase can have one or more amino acid variations at equivalent positions corresponding to the homologs of SEQ ID NO: 1, e.g., SEQ ID Nos 2-10 (Table 2).

TABLE 2

| | Analogs with | Analogs with | Analogs with |
|---|---|---|---|
| | larger, | smaller, | polar or |
| | hydrophobic | hydrophobic | charged starter |
| Position | starter molecules | starter molecules | molecules |
| G204 | A,C,P,V | A,C,P,V, L,I,M,F,W | S,T,Y,H,N,Q,D,E,K,R |
| G209 | A,C,P,V | A,C,P,V,L,I,M,F,W | S,T,Y,H,N,Q,D,E,K,R |
| D210 | A,C,P,V | A,C,P,V,L,I,M,F,W | S,T,Y,H,N,Q,E,K,R |
| G211 | A,C,P,V | A,C,P,V,L,I,M,F,W | S,T,Y,H,N,Q,D,E,K,R |
| G249 | A,C,P,V | A,C,P,V,L,I,M,F,W | S,T,Y,H,N,Q,D,E,K,R |
| G250 | A,C,P,V | A,C,P,V,L,I,M,F,W | S,T,Y,H,N,Q,D,E,K,R |
| F259 | G,A,C,P,V,L, | M,Y,W | S,T,Y,H,N,Q,D,E,K,R |
| | I,M,Y,W,S,T,H, | | |
| | N,Q,D,E,K,R | | |

In some embodiments the non-natural olivetol synthase has a higher affinity for butyryl-CoA as compared to the wild type olivetol synthase, wherein the amino acid sequence of the non-natural olivetol synthase comprises one or more amino acid substitutions at position(s) selected from the group consisting of A125S, A125T, A125C, A125Y, A125H, A125N, A125Q, A125W, A125F, A125V, S126R, S126N, S126D, S126C, S126Q, S126E, S126H, S126I, S126L, S126K, S126M, S126F, S126T, S126W, S126Y, S126V, D185G, D185Q, D185A, D185S, D185P, D185C, D185T, D185N, D185E, D185H, D185I, D185L, D185K, D185M, D185F, D185W, D185Y, D185V, M187H, M187F, M187Y, C189R, C189N, C189Q, C189H, C189I, C189L, C189K, C189M, C189F, C189T, L190Q, L190M, L190I, L190K, L190R, L190F, L190W, L190Y, F208Y, L257V, L257M, L257I, L257K, L257R, L257F, L257Y, L257H, L257P, F259V, F259L, F259I, F259M, F259W, F259T, F259Y, F259K, and F259R.

In embodiments wherein the non-natural olivetol synthase is based on a template that has less than 100% sequence identity to any one of SEQ ID NOs:1-10 (not including the particular variant or variant combinations described herein), those templates with less than 100% sequence identity can, in some embodiments, can have one or more amino acid changes from the template sequence at certain location(s), such as understood by alignment of two or more of SEQ ID NOs:1-10 to identify “variable positions.” For example, a non-natural non-natural olivetol synthase can include one, two, three, four, five, six, seven, eight, nine, ten, eleven, twelve, thirteen, fourteen, fifteen, sixteen, or seventeen amino acid changes at location(s) selected from the group consisting of position 25, 63, 75, 80, 81, 186, 187, 196, 198, 240, 258, 312, 315, 316, 375, 378, and 384, relative to SEQ ID NO:1, in addition to the one or more amino acid variations described herein, such as providing improved activity and/or selectivity. Exemplary amino acid changes at those positions include, but are not limited to, those as follows: 25I and 25L; 63I and 63C, 75K and 75R; 80D and 80E; 81V and 81M, 186I and 186M; 187M and 187T; 196E and 196D; 198D and 198N; 240I and 240E: 258I and 258M; 312H and 312D; 315S and 315K; 316D and 316E; 375R and 375T; 378V and 378L; and 384K and 384N.

In embodiments, the non-natural olivetol synthase can optionally be described with regards to “invariable amino acid(s),” which are those amino acid location(s) that are preferably not substituted in a template that has less than 100% sequence identity to any one of SEQ ID NOs:1-10 (not including the particular variant or variant combinations described herein). For example, in the non-natural non-natural olivetol synthase, some (50%, 60%, 70%, 80%, 85%, 90%, 93%, 95%, 97%, 98%, 99% or greater), or all (100%) of the following amino acids at the following locations do not vary from the referenced template sequence: 1M, 6A, 8G, 9P, 10A, 13L, 14A, 16G, 18A, 20P, 22N, 30P, 31D, 34F, 37T, 39S, 45L, 46K, 48K, 49F, 53C, 56S, 58I, 60K, 61R, 65L, 70L, 73N, 74P, 87R, 88Q, 92V, 96P, 97K, 98L, 100K, 102A, 106A, 107I, 108K, 109E, 110W, 111G, 113P, 115S, 117I, 118T, 119H, 126S, 130M, 132G, 140L, 141L, 142G, 143L, 145P, 149R, 151M, 152M, 153Y, 154Q, 156G, 157C, 160G, 162T, 164L, 165R, 167A, 168K, 169D, 171A, 172E, 173N, 174N, 176G, 177A, 178R, 179V, 180L, 191F, 192R, 194P, 202L, 203V, 204G, 208F, 209G, 210D, 211G, 212A, 214A, 215V, 216I, 217V, 218G, 221P, 227E, 229P, 244S, 246G, 248I, 250G, 251H, 256G, 257L, 259F, 263K, 264D, 265V, 266P, 268L, 272N, 273I, 277L, 289W, 290N, 294W, 297H, 298P, 299P, 300G, 302A, 303I, 304L, 307V, 310K, 313L, 317K, 321S, 322R, 325L, 326S, 329G, 330N, 331M, 332S, 333S, 336V, 338F, 341D, 344R, 346R, 347S, 349E, 352K, 354T, 356G, 358G, 360E, 361W, 362G, 364L, 366G, 367F, 368G, 369P, 370G, 372T, 373V, and 374E. For example, some of all of these invariable acids can be used in non-natural olivetol synthases one or more amino acid variation(s) selected from the group consisting of Q82S, P131A, I186F, M187S, S195K, S195M, S197V, T293E, K314D, and K314M. For non-natural olivetol synthases one or more amino acid variation(s) having one or more variations at position(s) 125, 126, 185, 187, 189, 190, 204, 208, 209, 210, 211, 249, 250, 257, 259, 331, and 332, the same invariable amino acids can be present with the exception of 126S, 204G, 208F, 209G, 210D, 211G, 250G, 257L, 259F, 331M, and 332S.

As used herein the term “non-naturally occurring”, when used in reference to an organism (e.g., microbial) is intended to mean that the organism has at least one genetic alteration not normally found in a naturally occurring organism of the referenced species. Naturally-occurring organisms can be referred to as “wild-type” such as wild type strains of the referenced species.

As used herein the term “non-naturally occurring” and “variant” and “mutant” are used interchangeably in the context of a polypeptide or nucleic acid. The term “non-naturally occurring” and “variant” in this context refers to a polypeptide or nucleic acid sequence having at least one variation at an amino acid position or a nucleic acid position as compared to a wild-type sequence.

Naturally-occurring organisms, nucleic acids, and polypeptides can be referred to as “wild-type” or “original” such as wild type strains of the referenced species. Likewise, amino acids found in polypeptides of the wild type organism can be referred to as “original” with regards to any amino acid position.

A genetic alteration that makes an organism non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

For example, in order to provide an olivetol synthase variant, an olivetol synthase from Cannabis sativa (NCBI Accession number AB164375; 385 amino acids long; SEQ ID NO: 1), can be selected as a template. Variants, as described herein, can be created by introducing into the template one or more amino acid substitutions to test for increased activity and improved specificity to 3,5,7-trioxododecanoyl-CoA, olivetol, or analogs thereof. In some cases, a “homolog” of the olivetol synthase SEQ ID NO: 1, is first identified. A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes that are orthologous can encode proteins with sequence similarity of about 45% to 100% amino acid sequence identity, and more preferably about 60% to 100% amino acid sequence identity. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

Genes sharing a desired amount of identify (e.g., 45%, 50%, 55%, or 60% or greater) to the Cannabis sativa BAG14339 olivetol synthase, including homologs, orthologs, and paralogs, can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor.

Computational approaches to sequence alignment and determination of sequence identity include global alignments and local alignments. Global alignment uses global optimization to forces alignment to span the entire length of all query sequences. Local alignments, by contrast, identify regions of similarity within long sequences that are often widely divergent overall. For understanding the identity of a target sequence to the Cannabis sativa BAG14339 olivetol synthase template a global alignment can be used. Optionally, amino terminal and/or carboxy-terminal sequences of the target sequence that share little or no identity with the template sequence can be excluded for a global alignment and generation of an identity score.

Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 45% to 100% sequence identity. Proteins that are unrelated can have an identity which is essentially the same as would be expected to occur by chance if a database of sufficient size is scanned (about 5%).

Pairwise global sequence alignment can be carried out using Cannabis sativa BAG14339 olivetol synthase SEQ ID NO: 1 as the template. Alignment can be performed using the Needleman-Wunsch algorithm (Needleman, S. & Wunsch, C. A general method applicable to the search for similarities in the amino acid sequence of two proteins J. Mol. Biol, 1970, 48, 443-453) implemented through the BALIGN tool (http://balign.sourceforge.net/). Default parameters are used for the alignment and BLOSUM62 was used as the scoring matrix. The disclosure also relates to Applicant's first discovery of wild-type sequences disclosed herein as an olivetol synthase and as having improved activity as also described herein; such wild-type sequences previously annotated as “hypothetical protein” or “putative protein.” Based in least on Applicant's identification, testing, motif identification, and sequence alignments (see FIG. 3), the current disclosure further allows for the identification of olivetol synthase suitable for use in engineered cells and methods of the disclosure, such as creating variants as described herein.

For the purpose of amino acid position numbering, SEQ ID NO: 1 is used as the reference sequence. For example, mention of amino acid position 49 is in reference to SEQ ID NO:1, but in the context of a different olivetol synthase sequence (a target sequence or other template sequence) the corresponding amino acid position for variant creation may have the same or different position number, (e.g. 48, 49 or 50). In some cases, the original amino acid and its position on the SEQ ID NO: 1 reference template will precisely correlate with the original amino acid and position on the target olivetol synthase. In other cases, the original amino acid and its position on the SEQ ID NO: 1 template will correlate with the original amino acid, but its position on the target will not be in the corresponding template position. However, the corresponding amino acid on the target can be a predetermined distance from the position on the template, such as within 10, 9, 8, 7, 6, 5, 4, 3, 2, or 1 amino acid positions from the template position. In other cases, the original amino acid on the SEQ ID NO: 1 template will not precisely correlate with the original amino acid on the target. However, one can understand what the corresponding amino acid on the target sequence is based on the general location of the amino acid on the template and the sequence of amino acids in the vicinity of the target amino acid, especially referring to the alignment provided in FIG. 3. It is understood that additional alignments can be generated with olivetol synthase sequences not specifically disclosed herein, and such alignments can be used to understand and generate new olivetol synthase variants in view of the current disclosure. In some modes of practice, the alignments can allow one to understand common or similar amino acids in the vicinity of the target amino acid, and those amino acids may be viewed as “sequence motif” having a certain amount of identity or similarity to between the template and target sequences. Those sequence motifs can be used to describe portions of olivetol synthase sequences where variant amino acids are located, and the type of variation(s) that can be present in the motif.

In some cases, it can be useful to use the Basic Local Alignment Search Tool (BLAST) algorithm to understand the sequence identity between an amino acid motif in a template sequence and a target sequence. Therefore, in preferred modes of practice, BLAST is used to identify or understand the identity of a shorter stretch of amino acids (e.g. a sequence motif) between a template and a target protein. BLAST finds similar sequences using a heuristic method that approximates the Smith-Waterman algorithm by locating short matches between the two sequences. The (BLAST) algorithm can identify library sequences that resemble the query sequence above a certain threshold. Exemplary parameters for determining relatedness of two or more sequences using the BLAST algorithm, for example, can be as set forth below. Briefly, amino acid sequence alignments can be performed using BLASTP version 2.0.8 (Jan. 05, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

FIG. 3 shows an alignment of SEQ ID NO: 1 (Cannabis sativa BAG14339) to other OLS homologs (SEQ ID NOs 2-10). These homologs were found by BLAST search, and range in sequence identity to SEQ ID NO: 1 from 52%-99% (SEQ ID NOs 2-10).

Methods known in the art can be used for the testing the enzymatic activity of OLS and OLS variant enzymes. As a general matter, an in vitro reaction composition will include an OLS or its variant (purified or in cell lysate or cell extract), malonyl-CoA, and an acyl-CoA (non-limiting examples include acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA, an aromatic acid CoA, for example, benzoic, chorismic, phenylacetic and phenoxyacetic acid CoA, or its analogs), and, in some embodiments, a purified OAC enzyme that can convert the substrates to the desired product, e.g., olivetolic acid or its analogs or derivatives, or a combination thereof, and in other embodiments, without OAC resulting in conversion of the substrates to olivetol, or its analogs or derivatives, or a combination thereof.

In some embodiments, the OAC enzyme is present in a non-rate limiting amount. In some embodiments, the OAC enzyme is present in a molar excess of the OLS enzyme. In other embodiments, the OAC enzyme is absent, or present in a rate limiting amount.

In some embodiments, at least a two-fold increase of enzymatic activity can be seen in in vitro reactions using cell lysates expressing olivetol synthase variants, or from purified preparations of the olivetol synthase variants (e.g., purified from cell lysates).

Cell lysis can be performed mechanically, such as by using a high pressure homogenizer or a bead mill, or non-mechanically. Non-mechanical methods include heating, osmotic shock, and cavitation (e.g., ultrasonic cavitation). Chemical methods include use of alkali conditions and detergents (e.g., SDS, Triton X TM, NP-40, Tween, CTAB, and CHAPS). Biological lysis materials include enzymes such as lysozyme lysostaphin, zymolase, cellulose, protease, and glycanase. In some embodiments, when using cell lysates, cells expressing olivetol synthase variants are treated by B-PERII™ reagent (ThermoFisher Scientific), in the presence of protease inhibitors, 10 mM DTT, benzonase and lysozyme. The lysate is added to the substrates comprising one or more acyl-CoA and malonyl-CoA in the presence or absence of purified OAC enzyme to initiate reactions. Reactions can run for 30 minutes before quenching with formic acid-acidified 75% acetonitrile. Samples can be centrifuged to remove cellular debris and then analyzed for the products formed using LCMS. Using a purified olivetol synthase preparation the rate of formation of can be determined. The rate can be expressed in terms of μM OLA/min/μM OLS. In some embodiments, the rate can be expressed in terms of μmol of OLA/min/ng of OLS or OL/min/ng of OLS. In some embodiments, the non-natural olivetol synthases in the presence of olivetolic acid cyclase provide a rate of formation of olivetolic acid, or without OAC provides a rate of formation of olivetol, of about 0.005 μM, 0.010 μM,0.020 μM, 0.050 μM, 0.100 μM, 0.250 μM, 0.500 μM, 1 μM, 1.5 μM, 2 μM, 2.5 μM, 3 μM, 3.5 μM, 4 μM, 4.5 μM, 5 μM, 5.5 μM, 6 μM or greater olivetolic acid or olivetol /min/μM enzyme.

Olivetolic acid cyclase (OAC), also known as polyketide cyclase, functions in concert with OLS/TKS to form olivetolic acid. The enzyme cyclizes the polyketides and has no intrinsic polyketide synthase activity. OAC requires the presence of OLS to produce olivetolic acid or its analogs. The OAC enzyme is classified as EC:4.4.1.26 under the enzyme commission nomenclature. Exemplary sequences of OAC are shown as SEQ ID NOs: 11 and 12. SEQ ID NO:12 is olivetolic acid cyclase (OAC) from Cannabis sativa, 101 aa, Accession Number XP_030508788.1); SEQ ID NO:11 is an OAC homolog, also 101 aa, and has an identity of 91% to SEQ ID NO:12.

In some embodiments, the amino acid sequence of olivetolic acid cyclase is at least 60%, 65%, 70%, 75%, 80%, 85%, 90%, 95%, 99%, or identical to at least 25, 30, 35, 40, 45, 50, 55, 60, 65, 70, 75, 80, 85, 90, 95 or more contiguous amino acids of any one of SEQ ID NO: 11 and SEQ ID NO: 12. In some embodiments, the amino acid sequence of olivetolic acid cyclase comprises one or more amino acid substitutions as compared to any one of SEQ ID NO: 11 and SEQ ID NO: 12. In some embodiments, the amino acid sequence of olivetolic acid cyclase is SEQ ID NO: 11 or SEQ ID NO: 12. In some embodiments, the amino acids His5, Ile7, Leu9, Phe23, Phe24, Tyr27, Val28, Leu30, Val40, Val59, Tyr72, Ile73, His78, Phe81, Gly82, Trp89, Leu92 and Ile94 corresponding to SEQ ID NO: 12 can be substituted with suitable amino acids.

In some embodiments wherein olivetolic acid, a derivative thereof, or an analog thereof is produced, the OAC is present in the engineered cell or in an in vitro reaction in a non-rate limiting amount or in a non-rate limiting enzymatic form. In some embodiments, the OAC is present in the engineered cell or in an in vitro reaction in molar excess of OLS. In some embodiments, the molar ratio of OLS to OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or more.

Site-directed mutagenesis or sequence alteration (e.g., site-specific mutagenesis or oligonucleotide-directed) can be used to make specific changes to a target olivetol synthase DNA sequence to provide a variant DNA sequence encoding olivetol synthase with the desired amino acid substitution. As a general matter, an oligonucleotide having a sequence that provides a codon encoding the variant amino acid is used. Alternatively, artificial gene synthesis of the entire coding region of the variant olivetol synthase DNA sequence can be performed as preferred olivetol synthase targeted for substitution are generally less than 400 amino acids long.

Exemplary techniques using mutagenic oligonucleotides for generation of a variant olivetol synthase sequence include the Kunkel method which may utilize an olivetol synthase gene sequence placed into a phagemid. The phagemid in E. coli produces olivetol synthase ssDNA which is the template for mutagenesis using an oligonucleotide which is a primer extended on the template.

Depending on the restriction enzyme sites flanking a location of interest in the olivetol synthase DNA, cassette mutagenesis may be used to create a variant sequence of interest. For cassette mutagenesis, a DNA fragment is synthesized inserted into a plasmid, cleaved with a restriction enzyme, and then subsequently ligated to a pair of complementary oligonucleotides containing the olivetol synthase variant mutation. The restriction fragments of the plasmid and oligonucleotide can be ligated to one another.

Another technique that can be used to generate the variant olivetol synthase sequence is PCR site directed mutagenesis. Mutagenic oligonucleotide primers are used to introduce the desired mutation and to provide a PCR fragment carrying the mutated sequence. Additional oligonucleotides may be used to extend the ends of the mutated fragment to provide restriction sites suitable for restriction enzyme digestion and insertion into the gene.

Commercial kits for site-directed mutagenesis techniques are also available. For example, the Quikchange™ kit uses complementary mutagenic primers to PCR amplify a gene region using a high-fidelity non-strand-displacing DNA polymerase such as pfu polymerase. The reaction generates a nicked, circular DNA which is relaxed. The template DNA is eliminated by enzymatic digestion with a restriction enzyme such as DpnI which is specific for methylated DNA.

An expression vector or vectors can be constructed to include one or more variant olivetol synthase encoding nucleic acids as exemplified herein operably linked to expression control sequences functional in the host organism. Expression vectors applicable for use in the microbial host organisms provided include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

As used herein the term “about” means within ±10% of the stated value. The term “about” can mean rounded to the nearest significant digit. Thus, about 5% means 4.5% to 5.5%. Additionally, “about” in reference to a specific number also includes that exact number. For example, about 5% also includes exact 5%.

As used herein, the term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism, the more than one exogenous nucleic acid(s) refers to the referenced encoding nucleic acid or biosynthetic activity, as discussed above. It is further understood, as disclosed herein, that more than one exogenous nucleic acid(s) can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

Exogenous variant olivetol synthase-encoding nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “isolated” when used in reference to a microbial organism is intended to mean an organism that is substantially free of at least one component that the referenced microbial organism is found with in nature. The term includes a microbial organism that is removed from some or all components as it is found in its natural environment. The term also includes a microbial organism that is removed from some or all components as the microbial organism is found in non-naturally occurring environments.

In some embodiments, the olivetol synthase variant gene is introduced into a cell with a gene disruption. The term “gene disruption,” or grammatical equivalents thereof, is intended to mean a genetic alteration that renders a target gene product inactive or attenuated. The genetic alteration can be, for example, deletion of the entire target gene, deletion of a regulatory sequence required for transcription or translation, deletion of a portion of the target gene which results in a truncated gene product, or by any of various mutation strategies that inactivate or attenuate the target gene product. One particularly useful method of gene disruption is complete gene deletion because it reduces or eliminates the occurrence of genetic reversions. The phenotypic effect of a gene disruption can be a null mutation, which can arise from many types of mutations including inactivating point mutations, entire gene deletions, and deletions of chromosomal segments or entire chromosomes. Specific antisense nucleic acid compounds and enzyme inhibitors, such as antibiotics, can also produce null mutant phenotype, therefore being equivalent to gene disruption.

A metabolic modification refers to a biochemical reaction that is altered from its naturally occurring state. Therefore, microorganisms may have genetic modifications to nucleic acids encoding metabolic polypeptides, or functional fragments thereof. Exemplary metabolic modifications are disclosed herein.

The microorganisms provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefmitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

A variety of microorganism may be suitable for incorporating the variant olivetol synthase, optionally with one or more other exogenous nucleic acid encoding one or more enzymes of the olivetolic acid pathway or cannabigerol pathway. Such organisms include both prokaryotic and eukaryotic organisms. In some embodiments, the eukaryotic microorganisms include, but are not limited to yeast, fungi, plant, or algae. In some embodiments, the eukaryotic microorganisms include microalgae.

Nonlimiting examples of microalgae for incorporating the non-natural olivetol synthase, optionally with one or more other exogenous nucleic acid encoding one or more enzymes of the olivetolic acid pathway or cannabigerol pathway include members of the genera Amphora, Ankistrodesmus, Aplanochytrium, Asteromonas, Boekelovia, Bolidomonas, Borodinella, Botrydium, Botryococcus, Bracteococcus, Carteria, Chaetoceros, Chlamydomonas, Chlorella, Chlorococcum, Chlorogonium, Chrococcidiopsis, Chroomonas, Chrysophyceae, Chrysosphaera, Colwellia, Cricosphaera, Oypthecodinium, Cryptococcus, Cryptomonas, Cunninghamella, Cyclotella, Desmodesmus, Dunaliella, Elina, Ellipsoidon, Emiliania, Eremosphaera, Ernodesmius, Euglena, Eustigmatos, Fragilaria, Fragilariopsis, Franceia, Gloeothamnion, Haematococcus, Hantzschia, Heterosigma, Hymenomonas, Isochrysis, Japanochytrium, Labrinthula, Labyrinthomyxa, Labyrinthula, Lepocinclis, Micractinium, Monodus, Monoraphidium, Moritella, Mortierella, Mucor, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Phaeodactylum, Phagus, Pichia, Picochlorum, Pithium, Platymonas, Pleurochrysis, Pleurococcus, Porphyridium, Prototheca, Pseudochlorella, Pseudoneochloris, Pseudostaurastrum, Pyramimonas, Pyrobotrys, Rhodosporidium, Scenedesmus, Schizochlamydella, Schizochytrium, Skeletonema, Spirulina, Spyrogyra, Stichococcus, Tetrachlorella, Tetraselmis, Thalassiosira, Thraustochytrium, Tribonema, Ulkenia, Vaucheria, Vibrio, Viridiella, Vischeria, and Volvox.

In some embodiments, the prokaryotic microorganisms include, but are not limited to bacteria, including archaea and eubacteria.

Exemplary microorganisms are reported in U.S. application Ser. No. 13/975,678 (filed Aug. 26, 2013), which is incorporated herein by reference in its entirety, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Thermus thermophilus, Pseudomonas species, including Pseudomonas aeruginosa, Pseudomonas putida, Pseudomonas stutzeri, Pseudomonas fluorescens, Rhodobacter spaeroides, Thermoanaerobacter brockii, Metallosphaera sedula, Leuconostoc mesenteroides, Chloroflexus aurantiacus, Roseiflexus castenholzii, Erythrobacter, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Haloferax mediterranei, Agrobacterium tumefaciens, Achromobacter denitrificans, Fusobacterium nucleatum, Streptomyces clavuligenus, Acinetobacter baumanii, Lachancea kluyveri, Trichomonas vaginalis, Trypanosoma brucei, Pseudomonas stutzeri, Bradyrhizobium japonicum, Mesorhizobium loti, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In certain embodiments, suitable organisms for incorporating the non-natural olivetol synthase include Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10 , Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476 , Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12 , Escherichia coli K-12 MG1655, Eubacterium hallii DSM 3353 , Flavobacterium frigoris, Fusobacterium nucleatum subsp. polymorphum ATCC 10953 , Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium , Salmonella typhimurium, Schizosaccharomyces pombe, Sebaldella termitidis ATCC 33386 , Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, and Yersinia intermedia.

FIG. 1 shows exemplary pathways to CBGA formation from malonyl-CoA, hexanoyl-CoA, and geranyl diphosphate. In some cases, the engineered cell of the disclosure can utilize hexanoyl-CoA that is produced from a cellular fatty acid biosynthesis pathway. For example, hexanoyl-CoA can be formed endogenously via reverse beta-oxidation of fatty acids.

In other embodiments, the engineered cell can further include one or more fatty acyl-CoA synthetase(s) which have broad substrate specificities, such as encoded by an exogenous nucleic acid(s). Exemplary fatty acyl-CoA synthetase genes, such as hexanoyl-CoA synthetase genes, include enzymes endogenous to bacteria, including E. coli, as well as eukaryotes, including yeast and C. sativa (see for example Stout et al., Plant J., 2012; 71:353-365, which is incorporated by reference in its entirety). Endogenous malonyl-CoA formation can be supplemented by formation from acetyl CoA using overexpression of acetyl-CoA carboxylase. Accordingly, the engineered cell can further include acetyl-CoA carboxylase, such as expressed on a transgene or integrated into the genome.

Acetyl-CoA carboxylase (EC 6.4.1.2) catalyzes the ATP-dependent carboxylation of acetyl-CoA to malonyl-CoA. This enzyme is biotin dependent and is the first reaction of fatty acid biosynthesis initiation in several organisms. Exemplary enzymes are encoded by accABCD of E. coli (Davis et al, J Biol Chem 275:28593-8 (2000)), ACCT of Saccharomyces cerevisiae and homologs (Sumper et al, Methods Enzym 71:34-7 (1981), which is incorporated by reference in its entirety).

FIG. 1 also shows prenyltransferase converts OLA and GPP to CBGA. Accordingly, the engineered cell can further include prenyltransferase, such as expressed on a transgene or integrated into the genome.

Optionally, the engineered cell can include one or more exogenous genes which allow the cell to grow on carbon sources the cell would not normally metabolize, or one or more exogenous genes or modifications to endogenous genes that allow the cell to have improved growth on carbon sources the cell normally uses. For example, WO2015/051298 (MDH variants) and WO2017/075208 (MDH fusions) describe genetic modifications that provide pathways allowing to cell to grow on methanol; WO2009/094485 (syngas) describes genetic modifications that provide pathways allowing to cell to grow on synthesis gas.

In some embodiments, the engineered cell may further comprise enzymes for geranyl phosphate pathways. For example, MVP pathway, MEP pathway, non-MVP, non-MEP pathways using isoprenol, prenol, and geraniol as precursors for the synthesis of geranyl pyrophosphate as disclosed in PCT application publication WO2017161041, which is incorporated by reference in its entirety.

As used herein, the term “conservative substitution” refers to conservatively modified variants. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

As used herein, the term “bioderived” means derived from or synthesized by a biological organism and can be considered a renewable resource since it can be generated by a biological organism. Such a biological organism, in particular the microbial organisms disclosed herein, can utilize feedstock or biomass, such as, sugars or carbohydrates obtained from an agricultural, plant, bacterial, or animal source. Alternatively, the biological organism can utilize atmospheric carbon. As. used herein, the term “biobased” means a product as described above that is composed, in whole or in part, of a bioderived compound of the disclosure. A biobased or bioderived product is in contrast to a petroleum derived product, wherein such a product is derived from or synthesized from petroleum or a petrochemical feedstock.

The cell cultures include engineered cells as disclosed herein that produce olivetolic acid, analogs and derivative of olivetolic acid and/or one or more cannabinoids or analogs or derivatives of the cannabinoids in a culture medium that includes a carbon source that can also be an energy source, such as glycerol, a sugar, a sugar alcohol, a polyol, an organic acid, or an amino acid. In various embodiments, the culture medium can include at least one feed molecule, including but not limited to, one or more organic acids, amino acids, or alcohols that can be converted into a precursor of a cannabinoid, cannabinoid analog, olivetolic acid, or an olivetolic acid precursor (e.g., acetyl-CoA, malonyl-CoA, hexanoyl-CoA, or other acyl-CoA molecules), or geranyldiphosphate).

Examples of feed molecules include, but are not limited to, bicarbonate, acetate, malonate, oxaloacetate, aspartate, glutamate, beta-alanine, alpha-alanine, a fatty acid (or its conjugate base, such as hexanoate, butyrate, pentanoate, heptanoate, octanoate, decanoate, etc.), a fatty alcohol (e.g., a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids, etc.), prenol, isoprenol and geraniol. Accordingly, “fatty acid” or “carboxylic acid” as used throughout herein includes acetate, propionate, butyrate, hexanoate, pentanoate, heptanoate, octonoate, decanoate, valerate, or isovalerate, a fatty acid of a chain length other than C6, a fatty acid of chain length C2-C22, including odd and even chain lengths, a C2, C4, C3, C5, C7, C8, C10, C12, C14, C16, C18, C20 or C22 chain length fatty acid, and an aromatic acid, for example benzoic, chorismic, phenylacetic and phenoxyacetic acids. Accordingly, “fatty alcohol” as used throughout herein includes a fatty alcohol of chain length C2-C22, a C2, C3, C4, C5, C7, C8, C10, C12, C14, C16, C18, C20 or C22 chain length fatty alcohol, ethanol, propanol, butanol, pentanol, hexanol, heptanol, octanol, decanol, dodecanol, tetradecanol, an aromatic alcohol, for example, benzyl alcohol and alcohols of chorismic, phenylacetic and phenoxyacetic acids, etc. In various embodiments, one, two, three, or more feed molecules can be present in the culture medium during at least a portion of the time the culture is producing olivetolic acid or a derivative thereof or a cannabinoid. Alternatively, or in addition, the culture medium can include a supplemental compound that can be a cofactor, or a precursor of a cofactor used by an enzyme that functions in a cannabinoid pathway, such as, for example, biotin, thiamine, pantothenate, or 4-phosphopantetheine. A culture medium in some embodiments can include one or more inhibitors of one or more enzymes, such as an enzyme that functions in fatty acid biosynthesis, such as but not limited to cerulenin, thiolactomycin, triclosan, diazaborines such as thienodiazaborine, isoniazid, and analogs thereof.

Further provided are methods for producing cannabinoids that include culturing a cell engineered for the production of olivetolic acid or a derivative thereof or a cannabinoid as provided herein under conditions in which the cell produces olivetolic acid, a derivative thereof, or a cannabinoid. In some examples, the methods include culturing the engineered cells in a culture medium that includes at least one feed molecule or supplement such as but not limited to: bicarbonate, acetate, malonate, oxaloacetate, aspartate, glutamate, beta-alanine, alpha-alanine, a fatty acid (or its conjugate base, such as hexanoate, butyrate, pentanoate, heptanoate, octanoate, nonanoate, decanoate, etc.), a fatty alcohol (includes a fatty alcohol of chain length C2-C22, a C2, C3, C4, C5, C7, C8, C9, C10, C12, C14, C16, C18, C20 or C22 chain length fatty alcohol, ethanol, propanol, butanol, pentanol, hexanol, heptanol, octanol, nonanol, decanol, dodecanol, tetradecanol, an aromatic alcohol, for example, benzyl alcohol and alcohols of chorismic, phenylacetic and phenoxyacetic acids), prenol, isoprenol, geraniol, biotin, thiamine, pantothenate, and 4-phosphopantetheine in the culture medium during at least a portion of the culture period when the cells are producing olivetolic acid, a derivative thereof, or a cannabinoid. Alternatively, or in addition, the methods can optionally include adding one or more fatty acid biosynthesis inhibitors to the culture medium during at least a portion of the culture period when the cells are producing olivetolic acid or a derivative thereof or a cannabinoid. The methods can further include recovering olivetolic acid or a derivative thereof or at least one cannabinoid from the cell, the culture medium, or whole culture. Also provided are cannabinoids produced by the methods provided herein, including derivatives of naturally-occurring cannabinoids, such as, but not limited to, cannabinoid derivatives having different acyl chain lengths than are found in naturally-occurring cannabinoids. The term “derivative” as used herein includes but is not limited to analogs.

In some embodiments, the cells provided herein that are engineered to produce olivetolic acid or a derivative thereof or a cannabinoid are further engineered to increase the production of the olivetolic acid, olivetolic acid derivative, or cannabinoid product, for example by increasing metabolic flux to a cannabinoid or olivetolic acid pathway, or by decreasing byproduct formation.

A cell engineered to produce olivetolic acid, an analog or derivative of olivetolic acid, or a cannabinoid, its analog or derivative is further engineered to increase the supply of coenzyme A (CoA) to increase its availability for producing acetyl-CoA and/or malonyl-CoA as well as hexanoyl-CoA or an alternative acyl-CoA.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, maltose, arabinose, cellobiose, lactose, and 3-, 4-, or 5-oligomers thereof. Other carbon sources include alcohol carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some embodiments, culture conditions include anaerobic or substantially anaerobic growth or maintenance conditions. Exemplary anaerobic conditions have been described previously and are well known in the art. Exemplary anaerobic conditions for fermentation processes are disclosed, for example, in U.S. Patent Application Publication No 2009/0047719, filed Aug. 10, 2007. Any of these conditions can be employed with the microbial organisms as well as other anaerobic conditions well known in the art.

The culture conditions can include, for example, liquid culture procedures as well as fermentation and other large scale culture procedures. Useful yields of the products can be obtained under aerobic, anaerobic or substantially anaerobic culture conditions.

An exemplary growth condition for achieving, one or more cannabinoid product(s) includes anaerobic culture or fermentation conditions. In certain embodiments, the microbial organism can be sustained, cultured or fermented under anaerobic or substantially anaerobic conditions. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

The culture medium at the start of fermentation may have a pH of about 5 to about 7. The pH may be less than 11, less than 10, less than 9, or less than 8. In other embodiments the pH may be at least 2, at least 3, at least 4, at least 5, at least 6, or at least 7. In other embodiments, the pH of the medium may be about 6 to about 9.5; 6 to about 9, about 6 to 8 or about 8 to 9.

Suitable purification and/or assays to test, e.g., for the production of 3-geranyl-olivetolate can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art.

The 3-geranyl-olivetolate (CBGA) or other target molecules may be separated from other components in the culture using a variety of methods well known in the art. Such separation methods include, for example, extraction procedures as well as methods that include liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. All of the above methods are well known in the art.

The disclosure also contemplates methods for, generally, forming an aromatic compound. The method involves contacting three molecules of malonyl-CoA and one molecule of acyl-CoA to form an aromatic compound. For example, in particular, the disclosure contemplates use of various acyl-CoA substrates such as acetyl-CoA, propionyl-CoA, butyryl-CoA, valeryl-CoA, hexanoyl-CoA, heptanoyl-CoA, nonanoyl-CoA, decanoyl-CoA, one or more of C12, C14, C16, C18, C20 or C22 chain length fatty acid CoA, an aromatic acid CoA, for example, benzoic, chorismic, phenylacetic and phenoxyacetic acid CoA in such an olivetol synthase-catalyzed reaction. The method can be performed in vivo (e.g., within the engineered cell) or in vitro.

The disclosure also contemplates methods for forming a prenylated aromatic compound. The method can be performed in vivo (e.g., within the engineered cell) or in vitro. In view of the improved specificity of the olivetol synthase variants, the disclosure also provides compositions that are enriched for the precursors for the desired cannabinoids, analogs and derivatives thereof, or combinations thereof.

In particular, the disclosure provides compositions enriched for olivetolic acid, analogs and derivatives of olivetolic acid. The nature of the olivetolic acid analogs will depend on the initial acyl-CoA substrate, e.g., acetyl-CoA, propionyl-CoA, butyryl-CoA, valeryl-CoA, hexanoyl-CoA, heptanoyl-CoA, octanoyl-CoA, nonanoyl-CoA, decanoyl-CoA, one or more of C12, C14, C16, C18, C20 or C22 chain length fatty acid CoA, an aromatic acid CoA, for example, benzoic, chorismic, phenylacetic and phenoxyacetic acid CoA.

The chemical structures and pathways for producing olivetolic acid and its analogs, cannabigerolic acid and its analogs, and cannabigerol and its analogs are shown in FIG. 5.

The olivetolic acid, analogs and derivatives of olivetolic acid can serve as a substrate for aromatic prenyltransferase and to produce cannabigerolic acid (CBGA) and its analogs and derivatives. CBGA and its analogs and derivatives can be decarboxylated either enzymatically, catalytically or thermally (by heat) to cannabigerol (CBG) and its analogs and derivatives.

As used herein, the terms “cannabinoid”, “cannabinoid product”, and “cannabinoid compound” or “cannabinoid molecule” are used interchangeably to refer a molecule containing a polyketide moiety, e.g., olivetolic acid or another 2-alkyl-4,6-dihydroxybenzoic acid, and a terpene-derived moiety e.g., a geranyl group. Geranyl groups are derived from the diphosphate of geraniol, known as geranyl-diphosphate or geranyl-pyrophosphate that forms the acidic cannabinoid cannabigerolic acid (CBGA). CBGA can be converted to further bioactive cannabinoids both enzymatically (e.g., by decarboxylation via enzyme treatment in vivo or in vitro to form the neutral cannabinoid cannabigerol), catalytically or thermally (e.g., by heating).

The term cannabinoid includes acid cannabinoids and neutral cannabinoids. The term cannabinoids also includes derivatives and analogs of naturally-occurring cannabinoids, such as, but not limited to, cannabinoids having different alkyl chain lengths of side groups than are found in naturally-occurring cannabinoids. The term “acidic cannabinoid” generally refers to a cannabinoid having a carboxylic acid moiety. The carboxylic acid moiety may be present in protonated form (i.e., as —COOH) or in deprotonated form (i.e., as carboxylate —COO⁻). Examples of acidic cannabinoids include, but are not limited to, cannabigerolic acid, cannabidiolic acid, and Δ⁹-tetrahydrocannabinolic acid. The term “neutral cannabinoid” refers to a cannabinoid that does not contain a carboxylic acid moiety (i.e., does contain a moiety —COOH or —COO⁻). Examples of neutral cannabinoids include, but are not limited to, cannabigerol, cannabidiol, and Δ⁹-tetrahydrocannabinol.

Cannabinoids may include, but are not limited to, cannabichromene (CBC), cannabichromenic acid (CBCA), cannabigerol (CBG), cannabigerolic acid (CBGA), cannabidiol (CBD), cannabidiolic acid (CBDA), Δ9-trans-tetrahydrocannabinol (Δ9-THC), Δ9-tetrahydrocannabinolic acid (THCA), Δ8-trans-tetrahydrocannabinol (Δ8 -THC), cannabicyclol (CBL), cannabielsoin (CBE), cannabinol (CBN), cannabinodiol (CBND), cannabitriol (CBT), cannabigerolic acid monomethylether (CBGAM), cannabigerol monomethylether (CBGM), cannabigerovarinic acid (CBGVA), cannabigerovarin (CBGV), cannabichromenic acid (CBCA), cannabichromevarinic acid (CBCVA), cannabichromevarin (CBCV), cannabidiol monomethylether (CBDM), cannabidiol-C4 (CBD-C4), cannabidivarinic acid (CBDVA), cannabidivarin (CBDV), cannabidiorcol (CBD-C1), Δ9-tetrahydrocannabinolic acid A (THCA-A), Δ9-tetrahydrocannabinolic acid B (THCA-B), Δ9-tetrahydrocannabinol (THC), Δ9-tetrahydrocannabinolic acid-C4 (THCA-C4), Δ9-tetrahydrocannabinol-C4 (THC-C4), Δ9-tetrahydrocannabivarinic acid (THCVA), Δ9-tetrahydrocannabivarin (THCV), Δ9-tetrahydrocannabiorcolic acid (THCA-C1), Δ9-tetrahydrocannabiorcol (THC-C1), Δ7-cis-iso-tetrahydrocannabivarin, Δ8-tetrahydrocannabinolic acid (Δ8-THCA), Δ8-tetrahydrocannabinol (Δ8-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

Cannabigerolic acid (CBGA) has the following chemical names (E)-3-(3,7-dimethyl-2,6-octadienyl)-2,4-dihydroxy-6-pentylbenzoic acid, and 3-[(2E)-3,7-dimethylocta-2,6-dien-1-yl]-2,4-dihydroxy-6-pentylbenzoic acid, and the following chemical structure:

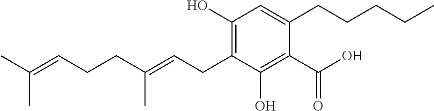 [SMILES: CCCCCc1cc(O)c(C/C=C(\C)CCC=C(C)C)c(O)c1C(=O)O]

Additional cannabinoid analogs and derivatives that can be produced with the methods or the engineered host cells of the present disclosure may also include, but are not limited to, 2-geranyl-5-pentyl-resorcylic acid, 2-geranyl-5-(4-pentynyl)-resorcylic acid, 2-geranyl-5-(trans-2-pentenyl)-resorcylic acid, 2-geranyl-5-(4-methylhexyl)-resorcylic acid, 2-geranyl-5-(5-hexynyl) resorcylic acid, 2-geranyl-5-(trans-2-hexenyl)-resorcylic acid, 2-geranyl-5-(5-hexenyl)-resorcylic acid, 2-geranyl-5-heptyl-resorcylic acid, 2-geranyl-5-(6-heptynoic)-resorcylic acid, 2-geranyl-5-octyl-resorcylic acid, 2-geranyl-5-(trans-2-octenyl)-resorcylic acid, 2-geranyl-5-nonyl-resorcylic acid, 2-geranyl-5-(trans-2-nonenyl) resorcylic acid, 2-geranyl-5-decyl-resorcylic acid, 2-geranyl-5-(4-phenylbutyl)-resorcylic acid, 2-geranyl-5-(5-phenylpentyl)-resorcylic acid, 2-geranyl-5-(6-phenylhexyl)-resorcylic acid, 2-geranyl-5-(7-phenylheptyl)-resorcylic acid, (6aR,10aR)-1-hydroxy-6,6,9-trimethyl-3-propyl-6a,7,8,10a-tetrahydro-6H-dibenzo[b,d]pyran-2-carboxylic acid, (6aR,10aR)-1-hydroxy-6,6,9-trimethyl-3-(4-methylhexyl)-6a,7,8,10a-tetrahydro-6H -dibenzo[b,d]pyran-2-carboxylic acid, (6aR,10aR)-1-hydroxy-6,6,9-trimethyl-3-(5-hexenyl)-6a,7,8,10a-tetrahydro-6H-dibenzo[b,d]pyran-2-carboxylic acid, (6aR,10aR)-1-hydroxy-6,6,9-trimethyl-3-(5-hexenyl)-6a,7,8,10a-tetrahydro-6H -dibenzo[b,d]pyran-2-carboxylic acid, (6aR,10aR)-1-hydroxy-6,6,9-trimethyl-3-(6-heptynyl)-6a,7,8,10a-tetrahydro-6H-dibenzo[b,d]pyran-2-carboxylic acid, 3-[(2E) -3,7-dimethylocta-2,6-dien-1-yl]-6-(hexan-2-yl)-2,4-dihydroxybenzoic acid, 3-[(2E) -3,7-dimethylocta-2,6-dien-1-yl]-2,4-dihydroxy-6-(2-methylpentypbenzoic acid, 3-[(2E)-3,7-dimethylocta-2,6-dien-1-yl]-2,4-dihydroxy-6-(3-methylpentyl)benzoic acid, 3-[(2E)-3,7-dimethylocta-2,6-dien-1-yl]-2,4-dihydroxy-6-(4-methylpentyl)benzoic acid, 3-[(2E)-3,7-dimethylocta-2,6-dien-1-yl]-2,4-dihydroxy -6-[(1E)-pent-1-en-1-yl]benzoic acid, 3-[(2E)-3,7-dimethylocta-2,6-dien-1-yl]-2,4-dihydroxy-6-[(2E)-pent-2-en-1-yl]benzoic acid, 3-[(2E)-3,7-dimethylocta-2,6-dien -1-yl]-2,4-dihydroxy-6-[(2E)-pent-3-en-1-yl]benzoic acid, 3-[(2E)-3,7-dimethylocta -2,6-dien-1-yl]-2,4-dihydroxy-6-(pent-4-en-1-yl)benzoic acid, 3-[(2E)-3,7-dimethylocta-2,6-dien-1-yl]-2,4-dihydroxy-6-propylbenzoic acid, 3-[(2E)-3,7-dimethylocta-2,6-dien-1-yl]-2,4-dihydroxy-6-butylbenzoic acid, 3-[(2E)-3,7-dimethylocta-2,6-dien-1-yl]-2,4-dihydroxy-6-hexylbenzoic acid, 3-[(2E)-3,7-dimethylocta-2,6-dien-1-yl]-2,4-dihydroxy-6-heptylbenzoic acid, 3-[(2E)-3,7-dimethylocta-2,6-dien-1-yl]-2,4-dihydroxy-6-octylbenzoic acid, 3-[(2E)-3,7-dimethylocta-2,6-dien-1-yl]-2,4-dihydroxy-6-nonanylbenzoic acid, 3-[(2E)-3,7-dimethylocta-2,6-dien-1-yl]-2,4-dihydroxy-6-decanylbenzoic acid, 3-[(2E)-3,7-dimethylocta-2,6-dien-1-yl]-2,4-dihydroxy-6-undecanylbenzoic acid, 6-(4-chlorobutyl)-3-[(2E)-3,7-dimethylocta-2,6-dien-1-yl]-2,4-dihydroxybenzoic acid, 3-[(2E)-3,7-dimethylocta-2,6-dien-1-yl]-2,4-dihydroxy-6-[4-(methylsulfanyl)butyl]benzoic acid, and others as listed in Bow, E. W. and Rimoldi, J. M., “The Structure-Function Relationships of Classical Cannabinoids: CB1/CB2 Modulation,” Perspectives in Medicinal Chemistry 2016:817-39 doi:10.4137/PMC.S32171, incorporated by reference herein.

Cannabinoid precursor analogs and derivatives that can be produced with the methods or genetically modified host cells of the present disclosure may also include, but are not limited to, divarinolic acid, 5-pentyl-resorcylic acid, 5-(4-pentynyl)-resorcylic acid, 5-(trans-2-pentenyl)-resorcylic acid, 5-(4-methylhexyl)-resorcylic acid, 5-(5-hexynyl)-resorcylic acid, 5-(trans-2-hexenyl)-resorcylic acid, 5-(5-hexenyl)-resorcylic acid, 5-heptyl-resorcylic acid, 5-(6-heptynoic)-resorcylic acid, 5-octyl-resorcylic acid, 5-(trans-2-octenyl)-resorcylic acid, 5-nonyl-resorcylic acid, 5-(trans-2-nonenyl)-resorcylic acid, 5-decyl-resorcylic acid, 5-(4-phenylbutyl)-resorcylic acid, 5-(5-phenylpentyl)-resorcylic acid, 5-(6-phenylhexyl)-resorcylic acid, and 5-(7-phenylheptyl)-resorcylic acid.

### Example 1: Structural Analysis

The online implementation of Rosetta from Cyrus Biotechnology was used to create homology models of crystal structures of OLS. The models used 1EE0 (2-pyrone synthase from Gerbera hybrida), 3OV2 (curcumin synthase from Curcuma longa), and 3AWK (polyketide synthase from Huperzia serrata) as the top three templates. Models are clustered by overall fold and the top scoring models from the five largest clustered are returned. These five models were highly similar to each other, signifying that the clusters converged toward one structure and giving confidence to an accurate model. The model from the largest cluster was used for analysis.

The model of OLS shares the same overall fold as other plant type 3 PKSs with known crystal structures, such as chalcone synthase (CHS) from Medicago sativa, for which there is much structural analysis in the literature. The catalytic triad of cysteine 157, histidine 297, and asparagine 330 as well as the ‘gatekeeper’ phenylalanine 208 (OLS numbering) are all present in OLS.

Aligning the OLS model with crystal structures containing bound ligands, specifically CHS structures 1CHW and 1CGZ, allowed for identification of OLS residues likely to interact with substrates. Manual analysis of the OLS model compared with literature information on the role of active site residues in other plant type III PKSs allowed for prediction of OLS active site residues' roles during catalysis. Residues were selected for contributing to one or more of three properties: starter molecule specificity, polyketide chain length, and cyclization reaction type. Starter molecule specificity refers to the initial substrate that binds in the active site and is elongated by the addition of extender molecules. For olivetolic acid, hexanoyl-CoA is the starter molecule and three malonyl-CoA are the extender molecules. Polyketide chain length refers to the number of ketide groups incorporated before cyclization. For olivetolic acid, the polyketide chain length is four (one from hexanoyl-CoA and three from the three malonyl-CoA molecules). Cyclization reaction type refers to the cyclization reaction that occurs among ketide groups to produce the final product. For olivetolic acid, the cyclization type is a C2 to C7 aldol condensation with retention of the terminal carboxyl group. It is hypothesized that the cyclization reaction to form olivetolic acid is performed by olivetolic acid cyclase (OAC). The final product of OLS (substrate of OAC) is unknown but it is hypothesized that it is most likely the linear tetraketide in free acid or CoA bound form or possibly the lactone formed by the C5-oxygen and C1 that then reopens before being cyclized by the OAC. In some embodiments, the cyclization reaction comprises cyclization of polyketides to olivetol analogs, derivatives, or combinations thereof by OLS by C2-C7 aldol condensation with C1 decarboxylation. The following residues were identified to play a role in starter molecule specificity, polyketide chain length preference and cyclization reaction.

The amino acid positions shown in the tables below of OLS corresponds to SEQ ID NO: 1. It is expressly contemplated that the amino acid sequence of the non-natural olivetol synthase can have one or more amino acid variations at equivalent positions corresponding to the homologs of SEQ ID NO: 1, e.g., SEQ ID Nos 2-10 (Table 3).

TABLE 3

| | Affect | Affect | Affect |
|---|---|---|---|
| | Starter | Polyketide | Cyclization |
| Posi- | Molecule | Chain | Reaction |
| tion | Specificity | Length | Type |
| A125 | | Yes | Yes |
| S126 | | Yes | |
| D185 | | Yes | |
| M187 | | Yes | |
| L190 | | Yes | |
| G204 | Yes | | |
| G209 | Yes | | |
| D210 | Yes | | |
| G211 | Yes | | |
| G249 | Yes | Yes | Yes |
| G250 | Yes | Yes | Yes |
| L257 | | | Yes |
| F259 | Yes | | Yes |
| M331 | | Yes | |
| S332 | | Yes | |

### Predicted Results of Amino Acid Substitutions

Residues predicted to contribute to starter molecule specificity interact with the starter molecule upon binding in the active site or after the catalytic cysteine has displaced the CoA portion of the starter molecule, so the focus will be on the non-CoA portion of the starter molecule. Both the size and biochemical properties of the starter molecule determine which mutations will increase specificity towards it. Large hydrophobic starter molecules such as CoA-bound aliphatic chains or aromatic rings will be bound better by amino acids with small hydrophobic side chains such as glycine, alanine, valine, leucine, isoleucine, or proline. Smaller hydrophobic starter molecules will thus be bound better by amino acids with large hydrophobic side chains such as methionine, phenylalanine, or tryptophan. Polar or charged starter molecules will benefit from amino acids with polar side chains such as serine, threonine, cysteine, tyrosine, histidine, glutamine, or asparagine as well as charged side chains such as aspartic acid, glutamic acid, lysine, and arginine.

Polyketide chain length is controlled through active site cavity volume. Substitutions at positions determining polyketide chain length with amino acids with larger side chains will result in reduced chain length. Substitution with amino acids with smaller side chains will result in extended chain length.

The means for predicting cyclization reaction type are not fully understood, but two controlling factors are known: positioning of the chain carbon atoms and ketone groups with respect to each other and the presence or absence of an ester bond at the C1 carboxylate. While the positioning of the chain carbon atoms and ketone groups with respect to each other is controlled by subtle interactions that cannot currently be accurately predicted, it is also controlled by active site volume in the cyclization pocket. A smaller volume allows less bending of the polyketide chain and thus fewer intramolecular interactions between the chain carbon atoms and ketone groups. Substitutions at positions determining cyclization reaction type with amino acids with larger side chains will result in reduced active site volume in this area and thus disfavor cyclization, leading to increased production of the linear tetraketide product. The presence of an ester bond at the C1 carboxylate highly favors a C6 to C1 Claisen condensation which would lead to a non-olivetolic acid product. The subtle hydrogen bond network throughout active site residues and water molecules that performs the cleavage of the C1-cysteine thioester bond and prevents Claisen condensation cannot be accurately modeled without a crystal structure of OLS. However, substitutions at positions determining cyclization reaction type with amino acids with polar side chains such as serine, threonine, cysteine, tyrosine, histidine, glutamine, or asparagine as well as charged side chains such as aspartic acid, glutamic acid, lysine, and arginine will promote formation of the necessary hydrogen bond network and should increase formation of the linear tetraketide.

The following amino acid substitutions are predicted to increase olivetolic acid production by OLS in the presence of OAC, or olivetol production in the absence of OAC (Table 4).

TABLE 4

| Position | Mutation |
|---|---|
| A125 | G,S,T,C,Y,H,N,Q,D,E,K,R |
| S126 | G,A |
| D185 | G,A,S,P,C,T,N |
| M187 | G,A,S,P,C,T,D,N,E,Q,H,V,L,I,K,R |
| L190 | G,A,S,P,C,T,D,N,E,Q,H,V,M,I,K,R |
| G204 | A,C,P,V,L,I,M,F,W |
| G209 | A,C,P,V |
| D210 | A,C,P,V |
| G211 | A,C,P,V |
| G249 | A,C,P,V,L,I,M,F,W,S,T,Y,H,N,Q,D,E,K,R |
| G250 | A,C,P,V,L,I,M,F,W,S,T,Y,H,N,Q,D,E,K,R |
| L257 | V,M,I,K,R,F,Y,W,S,T,C,H,N,Q,D,E |
| F259 | G,A,C,P,V,L,I,M,Y,W,S,T,Y,H,N,Q,D,E,K,R |
| M331 | G,A,S,P,C,T,D,N,E,Q,H,V,L,I,K,R |
| S332 | G,A |

The following amino acid substitutions at the positions are likely to affect the starter molecule specificity (G204, G209, D210, G211, G249, G250, and F259) and predicted to increase production of analog products by OLS using alternative starter molecules (Table 5).

TABLE 5

| | Analogs with | Analogs with | Analogs with |
|---|---|---|---|
| | larger, | smaller, | polar or |
| | hydrophobic | hydrophobic | charged starter |
| Position | starter molecules | starter molecules | molecules |
| G204 | A,C,P,V | A,C,P,V,L,I,M,F,W | S,T,Y,H,N,Q,D,E,K,R |
| G209 | A,C,P,V | A,C,P,V,L,I,M,F,W | S,T,Y,H,N,Q,D,E,K,R |
| D210 | A,C,P,V | A,C,P,V,L,I,M,F,W | S,T,Y,H,N,Q,E,K,R |
| G211 | A,C,P,V | A,C,P,V,L,I,M,F,W | S,T,Y,H,N,Q,D,E,K,R |
| G249 | A,C,P,V | A,C,P,V,L,I,M,F,W | S,T,Y,H,N,Q,D,E,K,R |
| G250 | A,C,P,V | A,C,P,V,L,I,M,F,W | S,T,Y,H,N,Q,D,E,K,R |
| F259 | G,A,C,P,V,L, | M,Y,W | S,T,Y,H,N,Q,D,E,K,R |
| | I,M,Y,W,S,T,, | | |
| | H,N,Q,D,E,K,R | | |

### Example 2: Library Constructs and Strains

Mutant variants of olivetol synthase were constructed as libraries on plasmid by single-site and multi-site (combinatorial) mutagenesis methods, using specific primers at the positions undergoing mutagenesis, amplifying fragments via PCR, and circularizing plasmid via Gibson ligation. For site-saturation mutagenesis of selected amino acids sites, a compressed-codon approach was used to eliminate codon redundancy to lower library size. For full-gene mutagenesis, a small set of codons representing amino acids Asp, Ala, Arg, and Phe (“DARF”) at each site in the gene were used. In cases where the wild-type amino acid is Asp, the set of amino acid substitution options was changed to Glu, Ala, Arg, or Phe. When the wild-type amino acid is Ala, the set of amino acid substitutions was changed to Asp, Gly, Arg, or Phe. When the wild-type amino acid is Arg, the set of amino acid substitutions was changed to Asp, Ala, Lys, or Phe. When the wild-type amino acid is Phe, the set of amino acid substitutions was changed to Asp, Ala, Arg, or Tyr. Plasmid used was the pZS* vector (Novagen), with expression of the olivetol synthase gene under control of a pA1 promoter and lac operator. The resulting olivetol synthase protein includes a fusion to a 6×Histidine tag at the N-terminus. Active variants were identified to activity assay described below and sequenced. Plasmids harboring the mutant libraries of olivetol synthase genes were transformed into an E. coli host with known thioesterase genes removed and plated onto Agar plates with suitable antibiotic selection.

### Cell Culture for Screening Homologs and Mutant Libraries

From both mutant library transformants and control transformants, single colonies were picked for growth into 384-well plates using Luria Bertani (LB) growth medium with carbenicillin. Following overnight growth, cultures were sub-cultured into fresh medium of LB with 1% glucose, carbenicillin, and IPTG. After 20 hours growth, cells were pelleted, and media discarded. Cells pellets were stored at −20 ° C. until ready for assay. Number of samples screened was approximately three times oversampling based on calculation of total possible variants.

### Example 3: High-Throughput Activity Assay

Cell pellets were thawed, then subjected to chemical lysis using B-PERII reagent in the presence of protease inhibitor cocktail, 10 mM DTT, benzonase, and lysozyme. Assays were performed in 384-well plates in a total volume of 50 μL, cell pellets were thawed, then subjected to chemical lysis using B-PERII reagent in the presence of protease inhibitor cocktail, 10 mM DTT, benzonase, and lysozyme. Assays were performed in 384-well plates in a total volume of 50-cultured into fresh mediu with malonyl-CoA synthetase, malonate and ATP. These enzymatic coupling reagents maintain malonyl-CoA in the assay by from free CoA generated by OLS catalysis. In some cases, purified OAC was included in the assays to generate the product OLA from the OLS intermediate tetraketide 3,5,7-trioxododeconoate.

Reactions were initiated by addition of cell lysate then incubated for 30 min; subsequently, 10 μl of reaction solution was removed and quenched into 15 volumes of 75% acetonitrile containing 0.1% formic acid and internal standards, then centrifuged to pellet denatured protein. Supernatants were transferred to new 384-well plates for LCMS analysis of olivetolic acid, olivetol, and PDAL.

### Analytical Analysis of OLS Reactions

Olivetol, PDAL, and OLA were quantified by LCMS or LCMS/MS methods using C18 reversed phase chromatography coupled to either Exactive (Thermofisher) or QTrap 4500 (Sciex) mass spectrometers.

Reversed phase LCMS was used, and compounds were identified by their LC retention times and MRM transitions specific to the compounds. LCMSMS analysis was conducted on Shimadzu UHPLC system coupled with AB Sciex QTRAP4500 mass spectrometer. Agilent Eclipse XDB C18 column (4.6×3.0 mm, 1.8 um) was used with a 1-min gradient elution at 1 mL/min using water containing 0.1% ammonia acetate as mobile phase A and 90% methanol containing 0.1% ammonia acetate as mobile phase B. The LC column temperature was maintained at 45° C. Negative ionization mode was used for all the analytes.

### Results

Under the screening conditions described above, products are detected in the low or sub μM range. For wild-type OLS reactions in the absence of OAC, significant products are OL and PDAL, and OLA is not a significant product. The desired product is OL, and the undesired (“derailment”) product is PDAL. A useful comparative measure of the effects of mutation on formation rates of product and by-product is the ratio relative to wild-type, hence (OL/PDAL)ₘᵤₜ÷(OL/PDAL)_{wr}. Results of formation of OL (rate) and OL/PDAL (selectivity) of the mutant relative to wild type are reflected using the “+”, “++”, “−”, “++”, and “n/c” indicators, reflecting the relative increases, decreases, or those showing no or negligible change (n/c). For wild-type OLS reactions in the absence of OAC, significant products are OL and PDAL, and OLA is not a significant product. Results are shown in Table 6.

TABLE 6

| | | Wild- | | Rate change | Selectivity |
|---|---|---|---|---|---|
| Amino | OLS | type | | relative | change |
| acid site | template | residue | Mutant | to WT | relative to WT |
| 82 | WT | Q | S | n/c | n/c |
| 131 | WT | P | A | n/c | + |
| 186 | WT | I | F | − | ++ |
| 187 | WT | M | E | − | + |
| 187 | WT | M | N | n/c | + |
| 187 | WT | M | T | n/c | + |
| 187 | WT | M | I | − | ++ |
| 187 | WT | M | S | n/c | ++ |
| 187 | WT | M | A | n/c | + |
| 187 | WT | M | L | − | ++ |
| 187 | WT | M | G | − | ++ |
| 187 | WT | M | V | − | ++ |
| 187 | WT | M | C | n/c | ++ |
| 187, 197 | WT | M, S | G, G | n/c | ++ |
| 195 | WT | S | K | n/c | + |
| 195 | WT | S | M | n/c | n/c |
| 195 | WT | S | R | − | + |
| 197 | M187S | S | V | n/c | ++ |
| 314 | WT | K | D | n/c | n/c |
| 314 | WT | K | M | + | n/c |

With reference to the data shown in Table 6, a number of variants generated by OLS mutagenesis demonstrated an overall rate decrease of PDAL formation. Screening experiments revealed several sites and certain residues at these sites that have the effect of lowering rate of PDAL formation while maintaining rate of OL formation. Measurement of PDAL decrease is reflected by the OL/PDAL ratio provided by the variant OLS relative to the wild type control. Measurement of PDAL decrease may also be reflected by the (OL+OLA)/PDAL ratio provided by the variant OLS relative to the wild type control when OAC is present.

The data shown in Table 6 also show a number of variants generated by OLS mutagenesis that demonstrated increase of OL formation. Screening experiments revealed sites and certain residues at these sites that have the effect of increasing the rate of product formation of OL.

### Example 4: Combination Mutants and Activity and Selectivity Assays

Based on results of the disclosure including the mutants described in Example 3, combination mutants were prepared; sequence verified, and then assayed for activity and selectivity in vitro using the procedures described in Example 3. Single mutants were selected from prior rounds of single-site screening and used to build double and triple mutants. Variants were screened in quadruplicate. OL and PDAL were measured for the activity assay. Activity and selectivity of the mutants were determined from the ratio to averaged controls (wild-type). Part of normalization procedure involved relative quantification of OLS via a split GFP fluorescence measurement. Results are shown in Tables 7-9.

TABLE 7
Single Variants based on SEQ ID NO:1

| | Rate change | Selectivity change |
|---|---|---|
| Variant | relative to WT | relative to WT |
| P131A | + | + |
| K314M | n/c | n/c |
| S197V | n/c | n/c |
| K314D | n/c | n/c |
| Q825 | n/c | n/c |
| M187S | n/c | + |
| T239E | n/c | n/c |
| S195K | − | + |
| I186F | − | + |
| S195M | − | + |

TABLE 8
Double Variants based on SEQ ID NO:1

| | Rate change | Selectivity change |
|---|---|---|
| Variant | relative to WT | relative to WT |
| Q82S, P131A | ++ | + |
| P131A, K314M | ++ | + |
| P131A, K314D | ++ | + |
| P131A, T239E | ++ | ++ |
| P131A, M187S | + | ++ |
| P131A, S197V | + | + |
| P131A, S195K | + | ++ |
| S195K, T239E | n/c | n/c |
| S195M, S197V | − | ++ |
| S195M, T239E | − | ++ |

TABLE 9
Triple Variants based on SEQ ID NO:1

| | Rate change | Selectivity change |
|---|---|---|
| Variant | relative to WT | relative to WT |
| Q82S, P131A, K314M | ++ | n/c |
| P131A, T239E, K314D | ++ | n/c |
| Q82S, P131A, K314D | ++ | n/c |
| Q82S, P131A, M187S | ++ | n/c |
| P131A, S197V, K314M | ++ | n/c |
| P131A, S197V, T239E | ++ | n/c |
| P131A, T239E, K314M | ++ | n/c |
| P131A, M187S, S197V | + | n/c |
| P131A, M187S, K314D | + | n/c |
| Q82S, P131A, T239E | + | n/c |
| P131A, S195M, K314M | + | ++ |
| P131A, M187S, T239E | + | n/c |
| P131A, S195M, K314D | + | + |
| P131A, S195K, K314D | n/c | + |
| P131A, S195K, K314M | n/c | + |
| P131A, M187S, S195M | n/c | ++ |
| S197V, T239E, K314M | n/c | + |
| Q82S, I186F, K314M | n/c | ++ |
| P131A, M187S, S195K | n/c | ++ |
| I186F, S197V, K314M | − | ++ |
| S195K, T239E, K314M | − | ++ |
| Q82S, I186F, S195M | − | ++ |
| I186F, S195K, K314M | − | + |
| I186F, S195M, K314D | − | ++ |
| I186F, S197V, K314D | − | ++ |
| I186F, M187S, K314M | − | + |
| S195K, S197V, K314M | − | ++ |
| I186F, S195K, K314D | − | + |
| I186F, S195M, T239E | − | ++ |
| Q82S, S197V, T239E | − | + |
| Q82S, I186F, K314D | − | n/c |
| I186F, S195M, K314M | − | + |
| S195K, S197V, T239E | − | ++ |
| S195M, S197V, T239E | − | ++ |
| S195M, S197V, K314M | − | ++ |
| S195K, S197V, K314D | − | ++ |
| I186F, M187S, S195K | − | ++ |

1. A non-natural olivetol synthase (OLS) comprising at least one amino acid variation as compared to a wild type olivetol synthase, wherein the non-natural olivetol synthase:
(a) forms olivetolic acid or olivetol from malonyl-CoA and hexanoyl-CoA at a greater rate as compared to the wild type olivetol synthase;
(b) has a higher affinity for hexanoyl-CoA and/or other acyl-CoA substrates as compared to the wild type olivetol synthase;
(c) forms olivetolic acid analogs, olivetol analogs, variants thereof, or combinations thereof from malonyl-CoA and other acyl-CoAs at a greater rate as compared to the wild type olivetol synthase;
(d) is characterized by a lower amount of one or more pyrone-based compounds being formed in the presence of the non-natural olivetol synthase (OLS) as compared to the wild type olivetol synthas, or
(e) any combination of (a), (b), (c) or (d),
wherein olivetolic acid or olivetol, analogs thereof, variants thereof, or acid derivatives of a polyketide are formed in the presence of olivetolic acid cyclase (OAC) not rate limited by amount or activity.

2. The non-natural olivetol synthase of claim 1, wherein
the pyrone-based hydrolysis compound is selected from pentyl diacetic acid lactone (PDAL), hexanoyl triacetic acid lactone (HTAL), and lactone analogs and derivatives thereof; or
the other acyl-CoA substrates are one or more of acetyl-CoA, propionyl-CoA, butyryl-CoA, valeryl-CoA, heptanoyl-CoA, octanoyl-CoA, nonanoyl-CoA, and decanoyl-CoA.

3-7. (canceled)

8. The non-natural olivetol synthase of claim 1, wherein:
(a) the non-natural olivetol synthase has lower affinity for 3,5,7 trioxododecyl-CoA, 3,5,7 trioxododecanoate, and analogs and derivatives thereof as substrates as compared to the wild type olivetol synthase, or optionally in the presence of non-natural olivetol synthase there is a lower rate of conversion of 3,5,7 trioxododecyl-CoA, 3,5,7 trioxododecanoate, analogs and derivatives thereof as substrates to pentyl diacetic acid lactone (PDAL) or hexanoyl triacetic acid lactone (HTAL), their analogs and derivatives thereof as compared to the wild type olivetol synthase;
(b) one or more pyrone-based compounds(s) are formed in a lower amount than the wild type olivetol synthase, and also capable of forming olivetolic acid or olivetol from malonyl-CoA and hexanoyl-CoA at a greater rate as compared to the wild type olivetol synthase and/or forming olivetolic acid analogs, olivetol analogs, variants thereof, or combinations thereof from malonyl-CoA and other acyl-CoAs at a greater rate as compared to the wild type olivetol synthase; or
(c) one or more pyrone-based hydrolysis product(s) are formed in an amount that is less than in the presence of the wild type olivetol synthase, and that provides a molar ratio of a polyketide or acid derivative thereof to the pyrone-based hydrolysis product(s) that is about 1.1-fold or greater, about 1.2-fold or greater, about 1.3-fold or greater, about 1.4-fold or greater, about 1.5-fold or greater, about 1.6-fold or greater, about 1.8-fold or greater, about 1.8-fold or greater, about 1.9-fold or greater, about 2.0-fold or greater, about 2.1-fold or greater, about 2.2-fold or greater, about 2.3-fold or greater, about 2.4-fold or greater, about 2.5-fold or greater, about 2.6-fold or greater, about 2.7-fold or greater, about 2.8-fold or greater, about 2.9-fold or greater, or about 3.0-fold or greater than the molar ratio in the presence of the wild type olivetol synthase.

9-11. (canceled)

12. The non-natural olivetol synthase of claim 13, wherein the non-natural olivetol synthase comprises at least two amino acid variations as compared to a wild type olivetol synthase, or at least three, four, five, or more amino acid variations as compared to a wild type olivetol synthase.

13. (canceled)

14. The non-natural olivetol synthase of claim 1, wherein
the wild type olivetol synthase comprises the amino acid sequence of any one of SEQ ID NOs: 1-10,
the amino acid sequence of the non-natural olivetol synthase has at least about 50%, 60%, 65%, 70%, 75%, 80%, 85%, 90%, 93%, 94%, 95%, 96%, 97%, 98%, or 99%, or greater sequence identity to at least 25 contiguous amino acids of any one of SEQ ID NOs: 1-10, or
the amino acid sequence of the non-natural olivetol synthase has at least about 90%, 93%, 94%, 95%, 96%, 97%, 98%, 99%, or greater identity to at least 25, 30, 35, 40, 50, 55, 60, 70, 75, 80, 90, 95, 100, 110, 120, 130, 140, 150, 160, 170, 180, 190, 200, 250, 300, 350, 355, 360, 365, 370, 375, or 380, or all, contiguous amino acids of any one of SEQ ID NOs:1-10.

15. (canceled)

16. (canceled)

17. The non-natural olivetol synthase of claim 14, wherein the amino acid sequence of the non-natural olivetol synthase comprises one or more amino acid variation(s) at position(s) selected from the group consisting of: Q82S, P131A, I186F, M187E, M187N, M187T, M187I, M187S, M187A, M187L, M187G, M187V, M187C, S195K, S195M, S195R, S197G, S197V, T239E, K314D, and K314M, corresponding to the amino acid positions of SEQ ID NO:1.

18. The non-natural olivetol synthase of claim 17,
comprising two, or more than two amino acid variations, selected from: (i) Q82S and P131A, (ii) Q82S and M187S, (iii) Q82S and S195K, (iv) Q82S and S195M, (v) Q82S and S197V, (vi) Q82S and K314D, (vii) P131A and I186F, (viii) P131A and M187S, (ix) P131A and S195M, (x) P131A and S197V, (xi) P131A and K314D, (xii) P131A and K314M, (xiii) I186F and M187S, (xiv) I186F and S195K, (xv) I186F and S195M, (xvi) I186F and T239E, (xvii) I186F and K314D, (xviii) M187S and S195K, (xix) M187S and S195M, (xx) M187S and S197V, (xxi) M187S and T239E, (xxii) M187S and K314D, (xxiii) M187S and K314M, (xxiv) S195K and S197V, (xxv) S195M and S197V, (xxvi) S195M and T239E, (xxvii) S195K and K314D, (xxviii) S195K and K314M, (xxix) S195M and K314D, (xxx) S195M and K314M, (xxxi) S197V and T239E, (xxxii) S197V and K314M, (xxxiii) T239E and K314D, (xxxiv) T239E and K314M, (xxxv) Q82S and I186F, (xxxvi) Q82S and T239E, (xxxvii) Q82S and K314M, (xxxviii) I186F and S197V (xxxix) I186F and K314M, (xl) S195K and T239E, (xli) S197V and K314D, (xlii) P131A and T239E, and (xliii) P131A and S195K.

19. The non-natural olivetol synthase of claim 17, comprising three, or more than three amino acid variations, selected from: (i) Q82S, P131A, and I186F, (ii) Q82S, P131A, and M187S, (iii) Q82S, P131A, and S195K, (iv) Q82S, P131A, and S195M, (v) Q82S, P131A, and S197V, (vi) Q82S, P131A, and T239E, (vii) Q82S, P131A, and K314D, (viii) Q82S, P131A, and K314M, (ix) Q82S, I186F, and M187S, (x) Q82S, I186F, and S195M, (xi) Q82S, I186F, and S197V, (xii) Q82S, I186F, and T239E, (xiii) Q82S, I186F, and K314D, (xiv) Q82S, I186F, and K314M, (xv) Q82S, M187S, and S195K, (xvi) Q82S, M187S, and S195M, (xvii) Q82S, M187S, and S197V, (xviii) Q82S, M187S, and T239E, (xix) Q82S, M187S, and K314D, (xx) Q82S, M187S, and K314M, (xxi) Q82S, S195K, and S197V, (xxii) Q82S, S195M, and S197V, (xxiii) Q82S, S195K, and K314D, (xxiv) Q82S, S195K, and K314M, (xxv) Q82S, S195M, and K314D, (xxvi) Q82S, S195M, and K314M, (xxvii) Q82S, S197V, and T239E, (xxviii) Q82S, S197V, and K314D, (xxix) Q82S, S197V, and K314M, (xxx) Q82S, T239E, and K314D, (xxxi) Q82S, T239E, and K314M, (xxxii) P131A, I186F, and M187S, (xxxiii) P131A, I186F, and S195K, (xxxiv) P131A, I186F, and S195M, (xxxv) P131A, I186F, and S197V, (xxxvi) P131A, I186F, and K314D, (xxxvii) P131A, I186F, and K314M, (xxxviii) P131A, M187S, and S195K, (xxxix) P131A, M187S, and S195M, (xl) P131A, M187S, and S197V, (xli) P131A, M187S, and T239E, (xlii) P131A, M187S, and K314D, (xliii) P131A, S195M, and S197V, (xliv) P131A, S195M, and T239E, (xlv) P131A, S195K, and K314D, (xlvi) P131A, S195K, and K314M, (xlvii) P131A, S195M, and K314D, (xlviii) P131A, S195M, and K314M, (xlix) P131A, S197V, and T239E, (1) P131A, S197V, and K314D, (1i) P131A, S197V, and K314M, (lii) P131A, T239E, and K314D, (liii) P131A, T239E, and K314M, (liv) I186F, M187S, and S195K, (1v) I186F, M187S, and S195M, (lvi) I186F, M187S, and S197V, (lvii) I186F, M187S, and K314M, I186F, S195K, and S197V, (lix) I186F, S195M, and S197V, (lx) I186F, S195K, and T239E, (lxi) I186F, S195M, and T239E, (lxii) I186F, S195K, and K314D, (lxiii) I186F, S195K, and K314M, (lxiv) I186F, S195M, and K314D, (lxv) I186F, S195M, and K314M, (lxvi) I186F, S197V, and T239E, (lxvii) I186F, S197V, and K314D, (lxviii) I186F, S197V, and K314M, (lxix) I186F, T239E, and K314M, (lxx) M187S, S195K, and S197V, (lxxi) M187S, S195M, and S197V, (lxxii) M187S, S195K, and T239E, (lxxiii) M187S, S195M, and T239E, (lxxiv) M187S, S195K, and K314D, (lxxv) M187S, S195K, and K314M, (lxxvi) M187S, S195M, and K314D, (lxxvii) M187S, S195M, and K314M, (lxxviii) M187S, S197V, and T239E, (lxxix) M187S, S197V, and K314D, (lxxx) M187S, S197V, and K314M, (lxxxi) M187S, T239E, and K314D, (lxxxii) M187S, T239E, and K314M, (lxxxiii) S195K, S197V, and T239E, (lxxxiv) S195M, S197V, and T239E, (lxxxv) S195K, S197V, and K314D, (lxxxvi) S195K, S197V, and K314M, (lxxxvii) S195M, S197V, and K314D, (lxxxviii) S195M, S197V, and K314M, (lxxxix) S195K, T239E, and K314D, (xc) S195K, T239E, and K314M, (xci) S195M, T239E, and K314D, (xcii) S195M, T239E, and K314M, and (xciii) S197V, T239E, and K314M.

20. The non-natural olivetol synthase of claim 14, wherein the amino acid sequence of the non-natural olivetol synthase comprises one or more amino acid variation(s) at position(s) selected from the group consisting of: 125, 126, 185, 187, 189, 190, 204, 208, 209, 210, 211, 249, 250, 257, 259, 331, and 332 corresponding to the amino acid positions of SEQ ID NO:1, wherein optionally the one or more amino acid variation(s) at position(s) are selected from the group consisting of: A125G, A125S, A125T, A125C, A125Y, A125H, A125N, A125Q, A125D, A125E, A125K, A125R, A125W, A125F, A125V, S126G, S126A, S126R, S126N, S126D, S126C, S126Q, S126E, S126H, S126I, S126L, S126K, S126M, S126F, S126T, S126W, S126Y, S126V, D185G, D185Q, D185A, D185S, D185P, D185C, D185T, D185N, D185E, D185H, D185I, D185L, D185K, D185M, D185F, D185W, D185Y, D185V, M187G, M187A, M187S, M187P, M187C, M187T, M187D, M187N, M187E, M187Q, M187H, M187V, M187L, M187I, M187K, M187R, M187F, M187Y, C189R, C189N, C189Q, C189H, C189I, C189L, C189K, C189M, C189F, C189T, L190G, L190A, L190S, L190P, L190C, L190T, L190D, L190N, L190E, L190Q, L190H, L190V, L190M, L190I, L190K, L190R, L190F, L190W, L190Y, G204A, G204C, G204P, G204V, G204L, G204I, G204M, G204F, G204W, G204S, G204T, G204Y, G204H, G204N, G204Q, G204D, G204E, G204K, G204R, F208Y, G209A, G209C, G209P, G209V, G209L, G209I, G209M, G209F, G209W, G209S, G209T, G209Y, G209H, G209N, G209Q, G209D, G209E, G209K, G209R, D210A, D210C, D210P, D210V, D210L, D210I, D210M, D210F, D210W, D210S, D210T, D210Y, D210H, D210N, D210Q, D210E, D210K, D210R, G211A, G211C, G211P, G211V, G211L, G211I, G211M, G211F, G211W, G211S, G211T, G211Y, G211H, G211N, G211Q, G211D, G211E, G211K, G211R, G249A, G249C, G249P, G249V, G249L, G249I, G249M, G249F, G249W, G249S, G249T, G249Y, G249H, G249N, G249Q, G249D, G249E, G249K, G249R, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, L257P, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M331I, M331K, M331R, S332G, and S332A corresponding to the amino acid positions of SEQ ID NO:1.

21-25.(canceled)

26. A nucleic acid encoding the non-natural olivetol synthase of any one of claim 1, the nucleic acid optionally being an expression construct wherein the nucleic acid encoding the non-natural olivetol synthase is operably linked to a regulatory element, wherein the regulatory element is heterologous to the olivetol synthase.

27. (canceled)

28. An engineered cell comprising a non-natural olivetol synthase of claim 1.

29. The engineered cell of claim 28, comprising enzymes for the olivetolic acid pathway, and optionally comprising olivetolic acid cyclase (OAC), optionally wherein OAC is at least 60% identical to at least 25 or more, or at least 95 or more contiguous amino acids of SEQ ID NO: 11 or SEQ ID NO: 12.

30-32. (canceled)

33. The engineered cell of claim 28, wherein the engineered cell comprises enzymes for the geranyl pyrophosphate pathway which optionally comprises geranyl pyrophosphate synthase, a mevalonate (MVA) pathway, a non-mevalonate (MEP) pathway, an alternative non-MEP, non MVA geranyl pyrophosphate pathway, or a combination of one or more pathways.

34. (canceled)

35. (canceled)

36. The engineered cell of claim 28, wherein the engineered cell comprises one or more exogenous nucleic acids, wherein at least one exogenous nucleic acid encodes the non-natural olivetol synthase, and optionally one or more exogenous nucleic acids enzymes for the geranyl pyrophosphate pathway.

37. (canceled)

38. (canceled)

39. The engineered cell of claim 28, wherein the cell is a prokaryote or a eukaryote.

40. (canceled)

41. The engineered cell of claim 39, wherein the cell is a prokaryote selected from the group consisting of Escherichia, Cyanobacteria, Corynebacterium, Bacillus, Ralstonia, and Staphylococcus.

42. (canceled)

43. A cell extract or cell culture medium of the engineered cell of claim 28 comprising olivetolic acid, cannabigerolic acid (CBGA), CBG, analogs or derivatives thereof, or a combination thereof, optionally wherein the cell extract or cell culture medium comprises olivetolic acid, analogs or derivatives thereof, or a combination thereof, at a concentration of 50% or greater of the total products of non-natural olivetol synthase catalyzed reactions.

44-46. (canceled)

47. A method for forming an aromatic compound, or a cannabinoid, an analog or derivatives thereof, or a combination thereof
where forming an aromatic compound comprises:
(a) contacting three molecules of malonyl-CoA and an acyl-CoA substrate with a non-natural olivetol synthase of claim 1, wherein the non-natural olivetol synthase preferentially produces polyketides, analogs, and derivatives thereof, or combinations thereof, over olivetol, analogs and derivatives of olivetol, pentyl diacetic acid lactone (PDAL), or lactone analogs and derivatives as compared to the wild type olivetol synthase;
(b) contacting the polyketides, analogs and derivatives thereof, or combinations thereof with a non-rate limiting amount of olivetolic acid cyclase (OAC) enzyme, wherein the contacting forms the aromatic compound; or
where forming a cannabinoid, an analog or derivatives thereof, or a combination thereof, comprises:
(a) contacting three molecules of malonyl-CoA and an acyl-CoA substrate with a non-natural olivetol synthase of claim 1, wherein the non-natural olivetol synthase preferentially produces polyketides, analogs, and derivatives thereof, or combinations thereof over olivetol, analogs and derivatives of olivetol, pentyl diacetic acid lactone (PDAL), or lactone analogs and derivatives as compared to the wild type olivetol synthase;
(b) contacting the polyketides, analogs and derivatives thereof, or combinations thereof with a non-rate limiting amount of olivetolic acid cyclase (OAC) enzyme, wherein the contacting forms the olivetolic acid, analogs and derivatives thereof, or combinations thereof;
(c) converting the olivetolic acid, analogs and derivatives thereof, or combinations thereof to the cannabinoid, an analog or derivatives thereof, or a combination thereof thermally, chemically or enzymatically, or by a combination thereof.

48-51. (canceled)

52. The method of claim 47, wherein the acyl-CoA substrate is selected from the group consisting of acetyl-CoA, propionyl-CoA, butyryl-CoA, valeryl-CoA, hexanoyl-CoA, heptanoyl-CoA, octanoyl-CoA, nonanoyl-CoA, and decanoyl-CoA.

53-57. (canceled)

58. A composition comprising a cannabinoid, analogs, or derivatives thereof, or combinations thereof obtained from the method of claim 49, wherein the composition comprises olivetol or analogs and derivatives of olivetol, pentyl diacetic acid lactone (PDAL), hexanoyl triacetic acid lactone (HTAL), a lactone analog, or a combination thereof at a concentration of no more than about 0.1% to about 0.0001% by weight of the composition.

59-62. (canceled)
